(19) United States
(12) Patent Application Publication
(10) Pub. No.: US 2007/0248978 A1
(43) Pub. Date: Oct. 25, 2007

(54) Steroid responsive nucleic acid expression and prediction of disease activity

(75) Inventors: Preeti Lal, Santa Clara, CA (US); Steven Rosenberg, Oakland, CA (US); Tod Klinger, San Carlos, CA (US)

Correspondence Address: MORRISON & FOERSTER LLP, 425 MARKET STREET, SAN FRANCISCO, CA, 94105-2482, US

(73) Assignee: Expression Diagnostics, Inc., Brisbane, CA (US)

(21) Appl. No.: 11/784,998

(22) Filed: Apr. 9, 2007

Related U.S. Application Data
(60) Provisional application No. 60/790,474, filed on Apr. 7, 2006.

(51) Int. Cl. C12Q 1/68; G06F 19/00; C12P 19/34

(52) U.S. Cl. 435/6; 435/91.2; 702/20

(57) ABSTRACT

The invention relates to methods useful for diagnosing and monitoring the steroid responsiveness of a subject by detecting expression of steroid modulated genes and for predicting transplant rejection and non-rejection.

### RELATED APPLICATION

This application claims priority to U.S. Patent Application No. 60/790,474, filed 7 Apr. 2006, which is incorporated by reference herein in its entirety.

### TECHNICAL FIELD

The invention relates to methods for detecting nucleic acid and protein expression modulated by steroids and using steroid responsiveness of a subject in predicting and monitoring disease activity.

### BACKGROUND OF THE INVENTION

Steroids are used to ameliorate disease activity associated with immune disorders such as graft rejection, systemic lupus erythematosis (SLE), multiple sclerosis (MS) and cytomegalovirus (CMV) infection. Although steroids are used clinically to treat hyperactivity of the immune system, prolonged treatment has deleterious effects including diabetes, osteoporosis and weight gain. Given these and other side effects, clinicians avoid prescribing high dosages of steroid any longer than necessary. Since flare of immune disorders and transplantation require the use of steroids as an ongoing treatment, it is desirable to determine the steroid responsiveness of a subject in order to optimize outcome. An essential component of providing effective immunosuppression is monitoring subject or transplant status. In transplant patients, this monitoring is organ, tissue or cell-specific. For example, monitoring a subject with a cardiac transplant involves taking a biopsy of heart muscle and having a pathologist examine it for cytological evidence of rejection. Such biopsies are expensive, invasive, and painful and interpretation can only be focused on the biopsied cells, not the whole organ.

Although glucocorticoid induction of genes correlated with immune response has been studied in vitro (Galon et al. (2002) FASEB Journal 16:61-71); there is a need for methods to detect in vivo expression of steroid modulated nucleic acids. The present invention addresses this need by diagnosing and monitoring steroid responsiveness or immunological status, predicting flares or graft rejection, and designing, evaluating or monitoring treatment efficacy.

### SUMMARY OF THE INVENTION

The present invention provides methods for detecting in vivo expression of nucleic acids and proteins modulated by steroid administration and metabolism. The invention presents a method of diagnosing or monitoring steroid responsiveness of a subject comprising detecting expression of a diagnostic set of at least two steroid modulated nucleic acids in a sample from the subject wherein the expression is correlated with steroid administration or dosage and applying at least one statistical method to the expression of the diagnostic set to diagnose or monitor steroid responsiveness of the subject.

In one embodiment, the diagnostic set further comprises at least one steroid modulated nucleic acid selected from each of at least two of the clusters of Table 1. In a second embodiment, the diagnostic set further comprises two or more steroid modulated nucleic acids selected from Table 2. In a third embodiment, the diagnostic set further comprises two or more steroid modulated nucleic acids selected from Table 3. In one aspect, detecting the expression of the diagnostic set of steroid modulated nucleic acids further comprises using hybridization or quantitative real-time polymerase chain reaction (RT-PCR) and a sample obtained from the subject by any sampling means. In a second aspect, the sample is a blood sample, and RNA is isolated from the peripheral blood mononuclear cells (PMBC) of the blood sample. In a third aspect, the statistical method is K-means clustering that produces clusters of genes that are correlated by p-value and their expression in a cell type or pathway or a prediction algorithm selected from a linear algorithm, a logistic regression algorithm, and a voting algorithm that produces a single value or score.

In a fourth embodiment, the diagnostic set further comprises selecting at least two oligonucleotides or a probe set to detect the expression of each steroid modulated nucleic acid of the diagnostic set. The invention also presents a kit comprising the oligonucleotides or probe sets that detect the expression of each steroid modulated nucleic acid of the diagnostic set. The invention further presents a method for diagnosing or monitoring steroid responsiveness of a subject comprising detecting the expression of nucleic acids encoding ADA, CD163, FKBP5, FLT3, FLT3LG, GZMA, IL1R1, IL1R2, ITGAM, NFKB1, PDCD1, THBS1, TNF, TRBC1 and TSC22D3.

The invention additionally presents a method for predicting rejection or non-rejection in a subject with a transplant comprising detecting expression of a diagnostic set of at least two steroid modulated nucleic acids in a sample from the subject wherein the expression of the steroid modulated nucleic acids correlates with transplant rejection or non-rejection, and applying at least one statistical method to the expression of the diagnostic set of steroid modulated nucleic acids to predict rejection or non-rejection.

In one embodiment, the diagnostic set of steroid modulated nucleic acids further comprises two or more nucleic acids selected from Tables 1-3. In one aspect, detecting the expression of the diagnostic set of steroid modulated nucleic acids further comprises using RT-PCR and RNA isolated from PMBCs. In a third aspect, the statistical method is a prediction algorithm selected from a linear algorithm, a logistic regression algorithm, and a voting algorithm that produces a single value or score that correlates with rejection or non-rejection. In a fourth aspect, the score that correlates with non-rejection is≦20 and the score that correlates with rejection is≧30. The invention yet further presents a method of predicting rejection or non-rejection comprising detecting the expression of a diagnostic set of steroid modulated nucleic acids encoding ADA, CD163, FKBP5, FLT3, FLT3LG, GZMA, IL1R1, IL1R2, ITGAM, NFKB1, PDCD1, THBS1, TNF, TRBC1 and TSC22D3.

The invention further presents a method of diagnosing or monitoring the status of a subject with a transplant comprising detecting expression of a diagnostic set of at least two steroid modulated nucleic acids in a sample from the subject wherein the expression is correlated with dysfunction or rejection of the transplant, and applying at least one statistical method to the expression of the nucleic acids to monitor the status of the transplant. In one embodiment, the diagnostic set further comprises two or more nucleic acids selected from Tables 1-3. In a second embodiment, RT-PCR is used with RNA isolated from PMBC to detect expression of the steroid modulated nucleic acids and the expression is analyzed using a prediction algorithm that produces single value or score that correlates with the status of the subject with the transplant. In a third embodiment, diagnosing and monitoring the status of a subject with a transplant further comprises detecting the expression of a diagnostic set of steroid modulated nucleic acids encoding ADA, CD163, FKBP5, FLT3, FLT3LG, GZMA, IL1R1, IL1R2, ITGAM, NFKB1, PDCD1, THBS1, TNF, TRBC1 and TSC22D3.

The invention also presents method for designing and monitoring a treatment plan for a subject with a transplant or an immune disorder comprising detecting expression of a diagnostic set of at least two steroid modulated nucleic acids in a sample from the subject wherein the expression correlates with the steroid responsiveness of the subject, and using the expression of the diagnostic set of steroid modulated nucleic acids to design and monitor the treatment plan of the subject. In one embodiment, the diagnostic set of steroid modulated nucleic acids comprises two or more nucleic acids selected from Tables 1-3. In a second embodiment, RT-PCR is used with RNA isolated from PMBC to detect expression of the steroid modulated nucleic acids and the expression is analyzed using a prediction algorithm that produces single value or score that correlates with the steroid responsiveness of the subject. In a third embodiment, diagnosing and monitoring the status of a subject with a transplant or immune disorder further comprises detecting the expression of a diagnostic set of steroid modulated nucleic acids encoding ADA, CD163, FKBP5, FLT3, FLT3LG, GZMA, IL1R1, IL1R2, ITGAM, NFKB1, PDCD1, THBS1, TNF, TRBC1 and TSC22D3 whose expression correlates with steroid responsiveness of a subject. In one aspect, the transplant is selected from bone marrow, heart, kidney, liver, lung, pancreas, pancreatic islets, stem cells, xenotransplants, and artificial implants. In another aspect, the immune disorder is selected from cytomegalovirus infection, multiple sclerosis, and systemic lupus erythematosus.

The invention yet still further presents a method for using primers and probe sets to detect steroid responsiveness of a subject with a transplant or an immune disorder comprising designing and generating primers or probe sets for nucleic acids whose expression is modulated by steroid administration or dosage, and using RT-PCR and the primers or probe sets on a sample from the subject to detect steroid responsiveness. In one embodiment, the nucleic acids whose expression is modulated by steroid administration or dosage are selected from Tables 1-3. In a second embodiment, the primers and probe sets are used in a diagnostic kit.

### BRIEF DESCRIPTION OF THE TABLES

Table 1 presents ten clusters of genes whose nucleic acid and protein expression is modulated by steroids. Column 1 shows cluster number; column 2, microarray probe ID from Human Genome CGH 44A Microarray (Agilent Technologies); column 3, gene symbol; column 4, average p-value for expression of the nucleic acid in CARGO and LARGO; column 5, average Pearson correlation for expression of the nucleic acid in CARGO and LARGO; column 6, p-value for the expression of the nucleic acid in CARGO, column 7, p-value for the expression of the nucleic acid in LARGO; and column 8, the name of the gene as it appears in the GenBank database (NCBI, Bethesda Md.).

Table 2 summarizes steroid modulated nucleic acid expression for 104 subject post-transplant samples and a subset of 74 samples≦180 days post-transplant. Column 1 shows the nucleic acids whose probe sets were used in RT-PCR to detect expression in post-transplant subject samples. The overall score refers to the single value produced from all scores using a linear discriminant algorithm. Columns 2-5 show the data for rejection (R) subjects, non-rejection (NR) subjects, the ratio, and p-values for all days post-transplant (index), respectively. Columns 6-9 show the data for rejection (R) subjects, non-rejection (NR) subjects, the ratio, and p-values for<180 days post transplant samples (subset), respectively. Significant p-values are shown in red typeface.

Table 3 presents RT-PCR data for 33 nucleic acids expressed in pathways having genes modulated by steroids or regulating T-cell homeostasis. Column 1 of Table 3 shows the gene symbol; columns 2 and 3, the fold change and p-value for R (n=38)/NR (n=55) at all times post-transplant; columns 4 and 5, the fold change and p-value for R (n=27)/NR (n=40) at≦180 days post-transplant; and column 6, the gene name.

### DETAILED DESCRIPTION OF THE INVENTION

The present invention addresses needs in the art by providing methods for detecting the in vivo expression of nucleic acids modulated by steroid administration or metabolism. The invention also provides methods for diagnosing and monitoring steroid responsiveness of a subject by detecting the expression of nucleic acids modulated by steroids. The invention uses detection of nucleic acids modulated by steroids to predict disease activity or transplant non-rejection or rejection and to determine status of an immune disorder or transplant. Such methods can be used to fine-tune immunosuppressant therapy and, more importantly, to reduce the number of invasive and costly tests and procedures that a subject must undergo. In particular, the invention can be used to predict transplant non-rejection or rejection. For example the invention can be used to predict transplant non-rejection or rejection allowing a clinician to reduce the number of biopsies performed in the first 180 days post-transplant or to begin anti-rejection therapy before cytological evidence of rejection is detectable. The invention also provides methods for evaluating the need for post-transplant monitoring and treatment or determining a subject's near-term prognosis based on steroid modulated nucleic acid expression.

Definitions

Unless defined otherwise, all scientific and technical terms are understood to have the same meaning as commonly used in the art to which they pertain. For the purpose of the present invention, the following terms are defined.

“Amplification” refers to any device, method or technique that can make copies of a nucleic acid. It can be achieved using a thermal cycler or a thermal gradient device and a polymerase chain reaction (PCR) technique such as linear amplification (cf. U.S. Pat. No. 6,132,997), rolling circle amplification, and the like. Further, amplification and detection can be combined as in Real-Time PCR (RT-PCR) using TAQMAN protocols and the Prism 7900HT Sequence Detection system and software (Applied Biosystems (ABI), Foster City Calif.).

“Array” refers to an ordered arrangement of at least two samples—nucleic acids, proteins or antibodies—in solution or on a substrate where at least one of the samples represents a control and/or normal sample and the other, a sample of diagnostic or prognostic interest. The ordered arrangement ensures that the size and signal intensity of each labeled complex, formed between at least one reagent and at least one sample to which the reagent specifically binds is readily detectable.

“Clusters” refers to groups of nucleic acids with expression that is directly or indirectly regulated by and correlated with the administration or metabolism of a steroid.

“Diagnostic set” refers to at least two nucleic acids whose expression is modulated by steroids and whose nucleic acids, oligonucleotides, primers and probe sets can be used in nucleic acid technologies or encoded proteins and antibodies or affinity reagents thereto can be used in protein technologies.

“Expression” refers to differential expression—increased or decreased expression as detected by presence, absence, or change in the amount of nucleic acid or protein expressed in a sample—as presented in a gene expression profile. A “gene expression profile” refers to the identification, characterization, quantification, and representation of a plurality of nucleic acids expressed in a sample from a subject as measured using nucleic acid or protein technologies. Nucleic acid expression is detected using nucleic acid technologies and mature mRNA transcript and/or regulatory sequences such as promoters, enhancers, introns, mRNA-processing intermediates, and 3′ untranslated regions. A gene expression profile from a subject can be compared with reference gene expression profiles based on detection of nucleic acid expression in control or normal, diseased, or treated samples.

“Immune disorders” refers to conditions, disorders and diseases associated with immunological response including but not limited to acute respiratory distress syndrome, Addison's disease, allograft rejection, ankylosing spondylitis, Takayasu's arteritis, arteriosclerosis, asthma, atherosclerosis, congestive heart failure, primary sclerosing cholangitis, Churg-Strauss syndrome, CREST syndrome, Crohn's disease, ulcerative colitis, diabetes mellitus, emphysema, glomerulonephritis, Wegener's granulomatosis, Grave's disease, autoimmune hepatitis, Kawasaki's syndrome, systemic lupus erythematosus, multiple sclerosis, myasthenia gravis, myelofibrosis, pancreatitis, polyarteritis nodosa, polymyositis, psoriasis, Raynaud's disease, Reiter's syndrome, rheumatoid arthritis, scleroderma, primary biliary sclerosis, systemic sclerosis, sepsis, septic shock syndrome, Sjogren's disease, ankylosing spondylitis, primary thrombocythemia, Hashimoto's thyroiditis, systemic vasculitis, Whipple's disease, complications of cancer, viral infection including CMV infection, bacterial infection, fungal infection, parasitic infection, protozoal infection, helminthic infection, and trauma.

“Immunosuppressant” refers to any therapeutic agent that suppresses immune response in a subject such as anticoagulents, antimalarials, heart drugs, non-steroidal anti-inflammatory drugs, and steroids including but not limited to aspirin, azathioprine, chloroquine, corticosteroids, cyclophosphamide, cyclosporin A, dehydroepiandrosterone, deoxyspergualin, dexamethasone, everolimus, fenoprofen, hydralazine, hydroxychloroquine, immunoglobulin, ibuprofen, indomethacin, leflunomide, ketoprofen, meclophenamate, mepacrine, 6-mercaptopurine, methotrexate, mizoribine, mycophenolate mofetil, naproxen, prednisone, methyprenisone, rapamycin (sirolimus), solumedrol, tacrolimus (FK506), thymoglobulin, tolmetin, tresperimus, triamcinoline, and the like.

“Monitoring” refers to repetitive testing for and detection of nucleic acid expression that provides useful information about a subject's health or disease status. Monitoring can include determining prognosis, risk-stratification, and efficacy of a particular drug; detecting subject response to a drug or ongoing therapy; predicting susceptibility, rejection or non-rejection, or disease activity; diagnosing onset, flare or complication of a disease; following disease progression or providing information related to a subject's status over time; selecting subjects most likely to benefit from a particular drug or experimental therapy especially where administration of that drug works for a small subset of subjects or where the drug does not have a label for a particular immune disorder; and screening a subject population to decide to use a more or less invasive or costly test; for example, moving from a non-invasive blood test to a more invasive option such as biopsy.

“Nucleic acid technology” refers to any and all devices, methods and systems used to detect expression of nucleic acids and produce a gene expression profile including but not limited to methods using arrays for hybridization, amplification in PCR, quantitative RT-PCR, TAQMAN protocol RT-PCR, multiplex PCR, thermal gradient devices, and the like, or hybridization in solution or on a substrate containing cDNAs, genomic DNAs, locked nucleic acids (LNAs), oligonucleotides, primers, peptide nucleic acids, polynucleotides, probe sets, RNAs and the like.

“Prediction” or “predicting” refers to the use of gene expression profile to provide information about a subject's health or the status of a disease, patient or transplant and can include determination of prognosis, risk-stratification, prediction of outcomes, and the like.

A “probe set” refers to groups of oligonucleotides or primers that can be used with a nucleic acid technology to detect groups of two or more nucleic acids. Primers in a probe set can contain rare or artificial nucleotides, be of any size useful in a nucleic acid technology, designed to detect a particular region or splice variant of a gene, labeled with one or more detectable moieties, and used in solution or attached to a substrate.

“Protein technology” refers to any and all devices, methods and systems that can be used to detect a peptide, polypeptide or protein expressed by a steroid modulated nucleic acid or gene and produce a gene expression profile including but not limited to activity assays, affinity assays, antibody or protein arrays, chromatographic separation, calorimetric assays, two-dimensional gel electrophoresis, ELISA, fluorescent-activated cell sorting, mass spectrophotometric detection, protein-fusion reporter constructs, western analysis, and the like. Protein expression, although time delayed, is correlated with and mirrors nucleic acid expression.

“Sample” is used in its broadest sense and refers to any biological material used for cytological or histological evaluation or to measure nucleic acid expression and obtained from a subject by any sampling means known to those of skill in the art. A sample can comprise a bodily fluid such as ascites, bile, blood, cerebrospinal fluid, synovial fluid, lymph, pus, semen, sputum, urine; the soluble fraction of a cell preparation, an aliquot of media in which cells were grown; a chromosome, an organelle, or membrane isolated or extracted from a cell; cDNA, genomic DNA, or RNA including but not limited to hnRNA, mRNA, mRNA processing intermediates, rRNA, and tRNA in solution or bound to a substrate; a cell; a cell, tissue or organ biopsy, and the like. Preferred samples for diagnosis, prognosis, or monitoring of immunological status are leukocytes, peripheral blood mononuclear cells (PBMC), or serum derived from whole blood.

“Sampling means” refers to any instrumentation and protocols for obtaining a biological sample and includes but is not limited to aspiration of a body fluid, aspiration of fluid following lavage, a biopsy (bronchoscopy or endoscopy) of cells, a tissue or organ, drawing of central or peripheral blood, and the like.

A “statistical method” refers to methods including but not limited to analysis of variance, canonical analysis, classification algorithms, classification and regression trees, cluster analysis including K-means clustering, factor analysis, Fisher's Exact test, k-nearest neighbor, linear algorithm, linear discriminatory analysis, linear regression, logistic algorithm, multidimensional scaling analysis, multiple regression, nearest shrunken centroids classifier, Pearson correlation, prediction algorithm, significance analysis of microarrays, one-tailed T-tests, two-tailed T-tests, voting algorithm, Wilcoxon's signed ranks test, and the like.

“Status” refers to any and all aspects of immune response in a subject who has an immune disorder or transplant including deterioration, improvement, progression, remission, or stability as determined from analyzing one or more samples from that subject for nucleic acid or protein expression that correlates with the degree and nature of response, steroid treatment or related complications including autoimmune cellular destruction, acute rejection, chronic rejection, humoral rejection, vasculopathy, and the like.

“Steroid modulated” refers to any gene product, nucleic acid or protein, whose expression is correlated with and results directly or indirectly from the administration or metabolism of steroids. For example, genes that have a steroid dependent regulatory element (sdre) in their promoter region (Dillner and Sanders (2002) J Biol Chem 277:33890-33894) are steroid modulated, primary response genes regulated by the presence and/or dosage of steroids. These primary response genes are often transcription factors that activate one or more indirectly affected, secondary response genes or pathways.

“Steroid responsive” or “steroid responsiveness” refers to any aspect of the immunological response of a subject to the administration or metabolism of steroids including improvement or worsening of symptoms, adjustment in dosage, change to another immunosuppressant, and the like.

“Subject” refers to an individual or patient who develops an infection, has an immune disorder or has received any allograft that elicits an immune response.

“Substrate” refers to any rigid or semi-rigid support to which antibodies, nucleic acids or proteins are bound and includes magnetic or nonmagnetic beads, capillaries or other tubing, chips, fibers, filters, gels, membranes, microparticles, plates, polymers, slides, and wafers with a variety of surface forms including channels, columns, pins, pores, trenches, wells and the like made from any natural or synthetic material or combination thereof.

“Transplant” refers to a subject's own genetically modified cells, or tissues grown from those cells; cells, tissues or organs from another subject or from an animal of a different species; and artificial implants such as mechanical or partially mechanical replacement organs.

“Transplant rejection” as detected or predicted using the methods and materials of the present invention refers to the rejection of bone marrow, heart, kidney, liver, lung, pancreas, pancreatic islets, stem cells, xenotransplants, and artificial implants.

“Quiescence” refers to the absence of signs or symptoms of histological or immunological response. For example, a diagnosis of remission in a subject with an immune disorder or non-rejection in a transplant patient indicates successful repression of immunological response and/or treatment with an immunosuppressant.

Description of the Invention

The correlation between the administration of steroids and the differential expression of steroid modulated nucleic acids and proteins provides an opportunity for developing pharmacogenomic markers for diagnosing and monitoring subjects with transplants, immune disorders such as SLE or MS, and CMV infection. As described in the Examples, the present invention provides methods, diagnostic sets of steroid modulated nucleic acids selected from Tables 1-3, and reagents such as antibodies, affinity reagents, primers and probe sets that can be used for determining, diagnosing, evaluating, monitoring, or predicting disease activity, non-rejection, rejection, status of a transplant or of an immune disorder, steroid responsiveness, and treatment plan of a subject with a transplant or immune disorder. In one embodiment, the ability to predict acute rejection can be used to begin immediate anti-rejection therapy while the ability to predict non-rejection can be used to determine the need for and timing of costly and invasive procedures such as biopsies. The invention additionally provides methods for designing and monitoring a treatment plan for a subject with an immune disorder or transplant and for evaluating the need for post-diagnosis or post-transplant monitoring and treatment.

The methods of the invention used RNA isolated from PBMC samples obtained from subjects enrolled in the Cardiac Allograft Rejection Gene Expression Observational (CARGO) and the Lung Allograft Rejection Gene Expression Observational (LARGO) studies. The samples were processed as described in Example 8 and used to study gene expression using nucleic acid technologies.

Microarray studies of gene expression were performed using the protocols described in Examples 9 and 10. These studies identified steroid modulated nucleic acids in the CARGO and LARGO samples from subjects treated with 1-100 mg doses of steroid as described in Example 1. Iterative cluster analysis and similarity testing as described in Example 4 were used to identify the nucleic acids modulated by steroids presented in Table 1. An exemplary RT-PCR study, carried out using the protocols described in Examples 13, used probe sets for 20 informative genes to investigate steroid responsiveness CARGO samples. The results of this study, as described in Example 5 and presented in Table 2, revealed that the expression of the nucleic acids known to be modulated by steroids were important both in diagnosing and monitoring steroid responsiveness and in predicting transplant rejection and non-rejection.

When the data from the exemplary RT-PCR study showed that differential expression of nucleic acids encoding FLT3, IL1R2, ITGAM, and PDCD1 proteins or fragments thereof was highly predictive of non-rejection within 180 days of transplant, a second study was performed to test additional nucleic acids in related pathways. Table 3 presents the results of the pathways RT-PCR study on 33 genes in the IL-1 or PDCD1 pathway, the ligand for FLT3, and genes induced and expressed in T cells. Using a p-value <0.05, the genes encoding ADA, CD163, FKBP5, FLT3, FLT3LG, GZMA, IL1R1, IL1R2, ITGAM, NFKB1, PDCD1, THBS1, TNF, TRBC1 and TSC22D3 protein and fragments thereof showed differential expression correlated with rejection.

Primers or probes sets that detect expression of at least one nucleic acid from the steroid modulated genes of Tables 1-3 can be used in a diagnostic set to carry out the methods of the invention. In one embodiment, the steroid modulated nucleic acids of the invention were used to design, select, and test primers and probe sets that can be used to detect steroid responsiveness in a sample from a subject as described in Examples 11 and 12. In another embodiment, antibodies or other affinity reagents specifically binding to a protein or a fragment thereof, expressed from steroid modulated genes of Tables 1-3, can be used in a diagnostic set to carry out the methods of the invention. Protein expression and antibody production and testing are described in Examples 15 and 16.

In a preferred embodiment, the methods and diagnostic sets of this invention can be used on clinically stable subjects, those showing no histological signs of rejection in endomyocardial biopsies (EMB) within 180 days of transplant to predict the probability that transplant rejection will occur within the subsequent 12 weeks. For example, a prediction algorithm was applied to the nucleic acid expression from exemplary RT-PCR studies to produce a single score for each subject. Then quartile analysis was applied to the single scores as described in Example 6. When used in longitudinal studies of≦180 days post transplant, the score produced by the algorithm distinguished clinically stable cardiac transplant subjects who did not reject their transplant in the subsequent 12 weeks, 98.9% with a score≦20, from those who progressed to acute cellular rejection, 58% with a score≧30.

Using a nucleic acid technology or a protein technology to generate a gene expression profile, one of skill in the art would select the appropriate devices and methods based upon such factors as the particular immune disorder or transplant, ease and needed accuracy of measurement of each particular nucleic acid or protein, the number of primers, probe sets or antibodies in the diagnostic set, and the like. It is contemplated that a gene expression profile based on a small diagnostic set of steroid modulated nucleic acids can be produced on a low density array or a thermal gradient chip in a clinic or a doctor's office.

Knowing steroid modulated in vivo expression of nucleic acids or proteins and establishing a correlation between their expression and steroid responsiveness, one of skill in the art can use diagnostic sets of primers, probe sets, antibodies and the like to determine the steroid responsiveness of a particular individual. To establish such correlations, nucleic acid or protein expression will be measured multiple times, and statistical methods or algorithms will be applied to determine the reliability of the measurement and to establish a threshold for the correlation. Correlations can be determined using samples from steroid responsive subjects. For example, knowing the steroid modulated in vivo expression levels of nucleic acids and an established correlation between the expression levels of such nucleic acids and the steroid responsiveness of a group of subjects being treated for transplant rejection, one of skill in the art can extrapolate the steroid responsiveness of a previously untested subject.

The responsiveness of a subject, based on nucleic acid expression, can be used design or to modify a treatment plan including types and amounts of immunosuppressants or steroids being administered; the dose, frequency and duration of treatment; weaning protocol, and the like. If a subject develops or shows resistance to a particular immunosuppressant, nucleic acid or protein expression and established correlations or profiles can be used to re-evaluate, the subject's responsiveness and to revise the subject's treatment plan.

Reagents used to establish a gene expression profile include but are not limited to genes and their splice variants, amplicons, LNAs, oligonucleotides, peptide nucleic acids, primers, and probe sets that can be used in nucleic acid technologies; and proteins and their fragments, antibodies, and affinity reagents that can be used in protein technologies. These reagents can be used in assays or diagnostic kits to determine or monitor steroid responsiveness of a subject, to screen or monitor subjects for the development or flare of immune disorder or for transplant rejection, to design or evaluate a treatment protocol, and the like.

Assays or diagnostic kits based on the reagents and nucleic acid and protein technologies described herein can be used with a sample from a subject to diagnose, classify or rule out an immune disorder such as SLE or MS; to select a clinical trial, to predict flare, to detect immunosuppressant or steroid responsiveness, to determine efficacy of a potential therapeutic agent, to design treatment regimes, to monitor the status of the subject or the treatment regime. In one alternative, the diagnostic kit comprises an array of reagents; in another, probe sets for use in RT-PCR.

Pharmacogenomics is the study of an individual's response to a particular therapeutic agent, immunosuppressant or combinations thereof. In this context, response refers to whether a particular drug will work better for a subject with a particular immune disorder or transplant. The methods disclosed provide for assigning a subject to a clinical trial or treatment regime based on disease or transplant status (quiescent or flare for immune disorder, rejection or non-rejection for transplant). Pharmacogenomics is also important in determining the dosage of a therapeutic agent based on age, classification and status of the subject. Individual steroid responsiveness, dosage and even timing of administration must be taken into account relative to side effects or potential interactions of various therapeutic agents. Some potentially useful therapeutic agents, immunosuppressants and steroids are listed in the definitions and/or claims.

All of the references cited are hereby incorporated by reference herein. This invention will be better understood by reference to the following non-limiting Examples which serve to demonstrate the use of nucleic acid and protein expression to evaluate steroid responsiveness in subjects, to optimize steroid dosage, to predict periods of non-rejection in subjects with transplants in order to reduce the number of invasive procedures, EMBs, TBBs, and the like.

Tables 1, 2 and 3 described in detail in the Examples are provided below.

TABLE 1

| | | | Average | | P value | | |
|---|---|---|---|---|---|---|---|
| Cluster | Probe Id | Gene Symbol | P value | Pearson | CARGO | LARGO | Gene Name |
| 1 | A_24_P146211 | HIST1H2BD | 0.000002 | 0.85 | 1.71E−08 | 0.000257 | histone 1, H2bd |
| 1 | A_23_P59069 | HIST1H2BO | 0.000006 | 0.86 | 1.77E−07 | 0.000175 | histone 1, H2bo |
| 1 | A_23_P366216 | HIST1H2BH | 0.000008 | 0.83 | 8.15E−08 | 0.00073 | histone 1, H2bh |
| 1 | A_24_P55148 | HIST1H2BJ | 0.000010 | 0.86 | 1.24E−06 | 8.55E−05 | histone 1, H2bj |
| 1 | A_23_P30776 | HIST1H2BE | 0.000010 | 0.85 | 2.64E−07 | 0.000406 | histone 1, H2be |
| 1 | A_23_P42178 | HIST1H2BF | 0.000012 | 0.85 | 7.09E−07 | 0.000195 | histone 1, H2bf |
| 1 | A_23_P402081 | HIST1H2BN | 0.000013 | 0.84 | 5.66E−07 | 0.000315 | histone 1, H2bn |
| 1 | A_24_P156911 | HIST2H2BE | 0.000015 | 0.86 | 8.42E−07 | 0.000269 | histone 2, H2be |
| 1 | A_23_P8013 | HIST1H2BL | 0.000019 | 0.84 | 3.65E−06 | 9.88E−05 | histone 1, H2bl |
| 1 | A_23_P111054 | HIST1H2BB | 0.000021 | 0.84 | 1.39E−06 | 0.000304 | histone 1, H2bb |
| 1 | A_23_P93180 | HIST1H2BC | 0.000023 | 0.8 | 3.31E−07 | 0.00159 | histone 1, H2bc |
| 1 | A_24_P152345 | LOC391566 | 0.000026 | 0.76 | 2.73E−08 | 0.0247 | Histone H2B.n |
| 1 | A_32_P57854 | | 0.000026 | 0.84 | 1.35E−06 | 0.0005 | DKFZp586A0722 |
| 1 | A_24_P3783 | HIST1H2BM | 0.000030 | 0.79 | 2.39E−07 | 0.00379 | histone 1, H2bm |
| 1 | A_23_P111041 | HIST1H2BI | 0.000031 | 0.8 | 5.68E−07 | 0.00166 | histone 1, H2bi |
| 1 | A_23_P30020 | PLA2G12A | 0.000050 | 0.86 | 1.45E−05 | 0.000173 | phospholipase A2, group XIIA |
| 1 | A_23_P218131 | C14orf151 | 0.000057 | 0.87 | 5.99E−05 | 5.34E−05 | chromosome 14 ORF 151 |
| 1 | A_23_P332992 | HIST3H2BB | 0.000059 | 0.84 | 9.35E−06 | 0.000368 | histone 3, H2bb |
| 1 | A_23_P256618 | C6orf79 | 0.000074 | 0.8 | 3.98E−06 | 0.00136 | chromosome 6 open reading |
| | | | | | | | frame 79 |
| 1 | A_24_P10884 | GRAP2 | 0.000074 | 0.76 | 2.09E−07 | 0.026 | GRB2-related adaptor protein 2 |
| 1 | A_23_P167997 | HIST1H2BG | 0.000079 | 0.78 | 2.72E−06 | 0.00229 | histone 1, H2bg |
| 1 | A_32_P100439 | Ells1 | 0.000086 | 0.88 | 3.41E−05 | 0.000215 | hypothetical protein Ells1 |
| 1 | A_24_P219785 | CALM3 | 0.000086 | 0.79 | 8.90E−07 | 0.00837 | calmodulin 3 |
| 1 | A_24_P164718 | MARCH2 | 0.000088 | 0.81 | 1.13E−05 | 0.000679 | membrane-associated ring finger |
| | | | | | | | (C3HC4) 2 |
| 1 | A_23_P154065 | TUBA1 | 0.000117 | 0.83 | 2.21E−05 | 0.000615 | tubulin, alpha 1 |
| 1 | A_23_P258093 | AGPAT1 | 0.000147 | 0.83 | 5.21E−05 | 0.000415 | 1-acylglycerol-3-phosphate O- |
| | | | | | | | acyltransferase 1 |
| 1 | A_23_P410312 | FLJ40142 | 0.000151 | 0.77 | 2.23E−06 | 0.0102 | FLJ40142 protein |
| 1 | A_23_P29124 | GP1BB | 0.000156 | 0.84 | 5.15E−05 | 0.000473 | glycoprotein Ib, beta polypeptide |
| 1 | A_23_P62351 | ARMCX6 | 0.000159 | 0.75 | 9.75E−07 | 0.0258 | armadillo repeat containing, X- |
| | | | | | | | linked 6 |
| 1 | A_23_P164047 | MMD | 0.000175 | 0.8 | 1.52E−05 | 0.00201 | monocyte to macrophage |
| | | | | | | | differentiation-associated |
| 1 | A_23_P33683 | MARCH2 | 0.000204 | 0.8 | 4.40E−05 | 0.00095 | membrane-associated ring finger |
| | | | | | | | (C3HC4) 2 |
| 1 | A_23_P154070 | TUBA1 | 0.000210 | 0.78 | 3.37E−05 | 0.00131 | tubulin, alpha 1 |
| 1 | A_23_P502710 | GAS2L1 | 0.000240 | 0.84 | 6.95E−05 | 0.000831 | growth arrest-specific 2 like 1 |
| 1 | A_32_P122754 | MGC17337 | 0.000259 | 0.79 | 9.01E−06 | 0.00746 | chromosome 9 ORF 30 |
| 1 | A_23_P128598 | TUBA2 | 0.000281 | 0.81 | 3.96E−05 | 0.00199 | tubulin, alpha 2 |
| 1 | A_23_P40470 | H2BFS | 0.000294 | 0.77 | 4.14E−06 | 0.0209 | H2B histone family, member S |
| 1 | A_23_P2114 | FLJ20625 | 0.000351 | 0.77 | 3.21E−05 | 0.00383 | hypothetical protein FLJ20625 |
| 1 | A_23_P366254 | SLC10A3 | 0.000431 | 0.78 | 3.30E−05 | 0.00562 | solute carrier family 10 member 3 |
| 1 | A_23_P103981 | HIST2H2AA | 0.000447 | 0.72 | 9.79E−06 | 0.0204 | histone 2, H2aa |
| 1 | A_23_P39684 | TLK1 | 0.000485 | 0.74 | 7.46E−06 | 0.0315 | tousled-like kinase 1 |
| 1 | A_24_P929650 | | 0.000499 | 0.68 | 3.37E−06 | 0.0739 | |
| 1 | A_23_P151120 | ACRBP | 0.000512 | 0.79 | 4.40E−05 | 0.00595 | acrosin binding protein |
| 1 | A_32_P61936 | | 0.000515 | 0.78 | 2.89E−05 | 0.00919 | clone IMAGE: 5173389 |
| 1 | A_24_P124957 | RAB11A | 0.000575 | 0.78 | 6.15E−05 | 0.00537 | RAB11A |
| 1 | A_23_P156550 | TREML1 | 0.000615 | 0.77 | 8.03E−05 | 0.00471 | triggering receptor expressed on |
| | | | | | | | myeloid cells-like 1 |
| 1 | A_32_P4814 | FAM11A | 0.000625 | 0.78 | 6.66E−05 | 0.00586 | family with sequence similarity |
| | | | | | | | 11 member A |
| 1 | A_23_P116264 | NRGN | 0.000664 | 0.76 | 3.83E−05 | 0.0115 | neurogranin |
| 1 | A_24_P10657 | CTL2 | 0.000725 | 0.75 | 0.000104 | 0.00505 | solute carrier family 44 member 2 |
| 1 | A_23_P330611 | WASPIP | 0.000828 | 0.75 | 0.00017 | 0.00403 | Wiskott-Aldrich syndrome |
| | | | | | | | protein interacting protein |
| 1 | A_23_P165840 | ODC1 | 0.000840 | 0.72 | 6.98E−05 | 0.0101 | ornithine decarboxylase 1 |
| 1 | A_23_P40718 | PARVB | 0.000912 | 0.68 | 1.68E−05 | 0.0495 | parvin, beta |
| 1 | A_23_P166677 | MFSD1 | 0.000996 | 0.76 | 0.000126 | 0.00788 | major facilitator superfamily |
| | | | | | | | domain containing 1 |
| 1 | A_23_P54488 | BG1 | 0.001028 | 0.75 | 5.34E−05 | 0.0198 | acyl-CoA synthetase bubblegum |
| | | | | | | | family member 1 |
| 1 | A_23_P78209 | MAFG | 0.001108 | 0.74 | 0.000307 | 0.004 | v-maf musculoaponeurotic |
| | | | | | | | fibrosarcoma oncogene homolog G |
| 1 | A_24_P74371 | PPGB | 0.001355 | 0.74 | 0.000157 | 0.0117 | protective protein for beta- |
| | | | | | | | galactosidase |
| 1 | A_24_P259490 | ARF1 | 0.001465 | 0.6 | 9.37E−06 | 0.229 | ADP-ribosylation factor 1 |
| 1 | A_23_P118038 | NUTF2 | 0.001469 | 0.73 | 0.000785 | 0.00275 | nuclear transport factor 2 |
| 1 | A_23_P98900 | FLJ22471 | 0.001471 | 0.73 | 0.000125 | 0.0173 | limkain beta 2 |
| 1 | A_23_P31177 | FLJ11000 | 0.001491 | 0.67 | 6.11E−05 | 0.0364 | hypothetical protein FLJ11000 |
| 1 | A_24_P74374 | PPGB | 0.001500 | 0.73 | 2.25E−05 | 0.1 | protective protein for beta- |
| | | | | | | | galactosidase |
| 1 | A_24_P825942 | | 0.001557 | 0.64 | 1.05E−05 | 0.231 | FLJ10934 fis |
| 1 | A_24_P107695 | ACTN1 | 0.001690 | 0.76 | 0.00121 | 0.00236 | actinin, alpha 1 |
| 1 | A_23_P147098 | MTPN | 0.001788 | 0.7 | 0.000283 | 0.0113 | myotrophin |
| 1 | A_32_P194848 | TAGLN2 | 0.002650 | 0.65 | 5.53E−05 | 0.127 | transgelin 2 |
| 1 | A_32_P75141 | | 0.003041 | 0.74 | 0.00108 | 0.00856 | |
| 1 | A_23_P76364 | CD9 | 0.003060 | 0.73 | 0.000466 | 0.0201 | CD9 antigen |
| 1 | A_23_P255444 | DAPP1 | 0.003114 | 0.61 | 3.20E−05 | 0.303 | dual adaptor of phosphotyrosine |
| | | | | | | | and 3-phosphoinositides |
| 1 | A_23_P102109 | TUBA4 | 0.003144 | 0.73 | 0.000308 | 0.0321 | tubulin, alpha 4 |
| 1 | A_24_P55465 | MTPN | 0.003519 | 0.64 | 7.94E−05 | 0.156 | myotrophin |
| 1 | A_23_P502224 | DIA1 | 0.003590 | 0.68 | 0.000651 | 0.0198 | cytochrome b5 reductase 3 |
| 1 | A_23_P100469 | TXNL4B | 0.003717 | 0.67 | 0.000225 | 0.0614 | thioredoxin-like 4B |
| 1 | A_23_P138717 | RGS10 | 0.003966 | 0.69 | 0.0011 | 0.0143 | regulator of G-protein signalling |
| | | | | | | | 10 |
| 1 | A_23_P162559 | SPPL3 | 0.004619 | 0.7 | 0.00983 | 0.00217 | signal peptide peptidase 3 |
| 1 | A_24_P137897 | IFRD1 | 0.004658 | 0.66 | 0.000716 | 0.0303 | interferon-related developmental |
| | | | | | | | regulator 1 |
| 1 | A_24_P147263 | USP31 | 0.005096 | 0.69 | 0.000757 | 0.0343 | ubiquitin specific peptidase 31 |
| 1 | A_23_P361773 | CCND3 | 0.005943 | 0.65 | 0.000678 | 0.0521 | cyclin D3 |
| 1 | A_23_P305711 | RYBP | 0.006042 | 0.68 | 0.000723 | 0.0505 | RING1 and YY1 binding protein |
| 1 | A_32_P192545 | LOC158931 | 0.006442 | 0.66 | 0.000497 | 0.0835 | transcription elongation factor A |
| | | | | | | | (SII)-like 6 |
| 1 | A_23_P141394 | WIPI49 | 0.007588 | 0.61 | 0.0236 | 0.00244 | WD repeat domain, |
| | | | | | | | phosphoinositide interacting 1 |
| 1 | A_23_P341392 | MGC32124 | 0.007797 | 0.67 | 0.0032 | 0.019 | hypothetical protein MGC32124 |
| 1 | A_23_P138881 | ACTN3 | 0.007808 | 0.64 | 0.00127 | 0.048 | actinin, alpha 3 |
| 1 | A_23_P434442 | TCEAL3 | 0.008260 | 0.67 | 0.00071 | 0.0961 | transcription elongation factor A |
| | | | | | | | (SII)-like 3 |
| 1 | A_23_P302550 | RGS18 | 0.009014 | 0.74 | 0.00549 | 0.0148 | regulator of G-protein signalling |
| | | | | | | | 18 |
| 1 | A_23_P30799 | HIST1H3F | 0.009138 | 0.62 | 0.0113 | 0.00739 | histone 1, H3f |
| 1 | A_24_P80135 | PTPN18 | 0.009658 | 0.62 | 0.000691 | 0.135 | protein tyrosine phosphatase, |
| | | | | | | | non-receptor type 18 |
| 1 | A_24_P319736 | MEIS1 | 0.010057 | 0.67 | 0.0165 | 0.00613 | myeloid ecotropic viral |
| | | | | | | | integration site 1 homolog |
| 1 | A_23_P6321 | CLDN5 | 0.011739 | 0.62 | 0.0432 | 0.00319 | claudin 5 |
| 1 | A_24_P186414 | TEX27 | 0.013616 | 0.5 | 0.000574 | 0.323 | zinc finger, AN1-type domain 3 |
| 1 | A_23_P215479 | CYLN2 | 0.015169 | 0.58 | 0.00133 | 0.173 | cytoplasmic linker 2 |
| 1 | A_23_P360379 | EGLN3 | 0.015179 | 0.57 | 0.00144 | 0.16 | egl nine homolog 3 |
| 1 | A_23_P95470 | CD151 | 0.016468 | 0.54 | 0.000565 | 0.48 | CD151 antigen |
| 1 | A_24_P29733 | PFTK1 | 0.021128 | 0.59 | 0.0036 | 0.124 | PFTAIRE protein kinase 1 |
| 1 | A_24_P333525 | RABGAP1L | 0.021667 | 0.56 | 0.00144 | 0.326 | RAB GTPase activating protein |
| | | | | | | | 1-like |
| 1 | A_23_P200325 | RABGAP1L | 0.024429 | 0.55 | 0.0016 | 0.373 | RAB GTPase activating protein |
| | | | | | | | 1-like |
| 1 | A_23_P502915 | WDR1 | 0.024880 | 0.52 | 0.00259 | 0.239 | WD repeat domain 1 |
| 1 | A_23_P132226 | TPST2 | 0.025788 | 0.56 | 0.0025 | 0.266 | tyrosylprotein sulfotransferase 2 |
| 1 | A_24_P922357 | LOC128977 | 0.027026 | 0.53 | 0.00658 | 0.111 | hypothetical protein LOC128977 |
| 1 | A_23_P141688 | RAB31 | 0.029041 | 0.58 | 0.0451 | 0.0187 | RAB31 |
| 1 | A_24_P134834 | DKFZp547E052 | 0.030231 | 0.49 | 0.0037 | 0.247 | hypothetical protein LOC84236 |
| 1 | A_24_P179339 | | 0.030626 | 0.55 | 0.0111 | 0.0845 | humanin |
| 1 | A_23_P126135 | MFN2 | 0.030828 | 0.54 | 0.0337 | 0.0282 | mitofusin 2 |
| 1 | A_24_P244916 | SERF2 | 0.032581 | 0.47 | 0.00386 | 0.275 | small EDRK-rich factor 2 |
| 1 | A_23_P141974 | TPM4 | 0.032790 | 0.54 | 0.0048 | 0.224 | tropomyosin 4 |
| 1 | A_23_P251825 | IFRD1 | 0.032850 | 0.6 | 0.0188 | 0.0574 | interferon-related developmental |
| | | | | | | | regulator 1 |
| 1 | A_23_P48175 | MGC5576 | 0.035875 | 0.57 | 0.055 | 0.0234 | transmembrane protein 106C |
| 1 | A_32_P119165 | | 0.037532 | 0.57 | 0.0156 | 0.0903 | |
| 1 | A_32_P42780 | | 0.041711 | 0.51 | 0.0578 | 0.0301 | |
| 1 | A_23_P502913 | WDR1 | 0.041857 | 0.54 | 0.02 | 0.0876 | WD repeat domain 1 |
| 1 | A_23_P27207 | SCGB1C1 | 0.045425 | 0.49 | 0.114 | 0.0181 | secretoglobin, family 1C member 1 |
| 1 | A_24_P403303 | PHF20L1 | 0.046385 | 0.5 | 0.0132 | 0.163 | PHD finger protein 20-like 1 |
| 1 | A_24_P316059 | | 0.051220 | 0.55 | 0.045 | 0.0583 | |
| 1 | A_23_P147199 | ZNF271 | 0.066915 | 0.4 | 0.0116 | 0.386 | zinc finger protein 271 |
| 1 | A_32_P55979 | | 0.080677 | 0.45 | 0.0576 | 0.113 | 6-pyruvoyltetrahydropterin |
| | | | | | | | synthase |
| 1 | A_32_P163089 | LOC387882 | 0.095436 | 0.45 | 0.012 | 0.759 | hypothetical protein |
| 1 | A_23_P2661 | RAP1B | 0.382999 | 0.26 | 0.384 | 0.382 | RAP1B |
| 1 | A_23_P154294 | MGC13005 | 0.440704 | −0.12 | 0.195 | 0.996 | FLJ44010 fis |
| 2 | A_23_P210330 | | 0.000031 | 0.88 | 2.72E−05 | 3.45E−05 | CS0DL009YB17 of B cells |
| 2 | A_24_P365901 | MGC50844 | 0.000038 | 0.87 | 2.91E−06 | 0.000507 | tetraspanin 33 |
| 2 | A_24_P226322 | SH3BGRL2 | 0.000048 | 0.85 | 6.74E−06 | 0.000337 | SH3 domain binding glutamic |
| | | | | | | | acid-rich protein like 2 |
| 2 | A_23_P152906 | ALOX12 | 0.000071 | 0.86 | 8.47E−06 | 0.000587 | arachidonate 12-lipoxygenase |
| 2 | A_24_P148321 | HIST2H2BE | 0.000071 | 0.77 | 6.88E−07 | 0.00738 | histone 2, H2be |
| 2 | A_23_P256205 | ABLIM3 | 0.000074 | 0.83 | 8.81E−06 | 0.000629 | actin binding LIM protein family |
| | | | | | | | member 3 |
| 2 | A_24_P209171 | SH3BGRL2 | 0.000081 | 0.84 | 1.39E−05 | 0.000471 | SH3 domain binding glutamic |
| | | | | | | | acid-rich protein like 2 |
| 2 | A_23_P390006 | PCSK6 | 0.000134 | 0.77 | 5.81E−06 | 0.00309 | proprotein convertase |
| | | | | | | | subtilisin/kexin type 6 |
| 2 | A_23_P129221 | FAH | 0.000137 | 0.7 | 1.83E−07 | 0.102 | fumarylacetoacetate hydrolase |
| 2 | A_23_P430818 | HSPC159 | 0.000152 | 0.82 | 0.000114 | 0.000202 | HSPC159 protein |
| 2 | A_24_P218905 | NET-5 | 0.000162 | 0.8 | 4.72E−05 | 0.000556 | tetraspanin 9 |
| 2 | A_32_P145477 | | 0.000218 | 0.84 | 5.04E−05 | 0.000947 | BX350256 |
| 2 | A_24_P290188 | | 0.000233 | 0.81 | 0.000152 | 0.000357 | |
| 2 | A_24_P706752 | | 0.000244 | 0.83 | 4.95E−05 | 0.0012 | |
| 2 | A_23_P143720 | GRAP2 | 0.000276 | 0.77 | 4.55E−06 | 0.0168 | GRB2-related adaptor protein 2 |
| 2 | A_23_P77971 | ITGA2B | 0.000315 | 0.78 | 2.23E−05 | 0.00445 | integrin, alpha 2b |
| 2 | A_23_P212436 | CTDSPL | 0.000394 | 0.82 | 3.79E−05 | 0.0041 | carboxy-terminal domain, RNA |
| | | | | | | | polymerase II, polypeptide A |
| 2 | A_23_P152926 | GP1BA | 0.000398 | 0.85 | 0.00144 | 0.00011 | glycoprotein Ib, alpha |
| | | | | | | | polypeptide |
| 2 | A_24_P189997 | PCSK6 | 0.000403 | 0.69 | 4.78E−06 | 0.034 | proprotein convertase |
| | | | | | | | subtilisin/kexin type 6 |
| 2 | A_23_P38519 | ITGB3 | 0.000408 | 0.81 | 0.000111 | 0.0015 | integrin, beta 3 |
| 2 | A_24_P64167 | PTGS1 | 0.000411 | 0.78 | 3.30E−05 | 0.00513 | prostaglandin-endoperoxide |
| | | | | | | | synthase 1 |
| 2 | A_24_P318656 | ITGB3 | 0.000422 | 0.83 | 0.000323 | 0.000552 | integrin, beta 3 |
| 2 | A_23_P2414 | | 0.000447 | 0.73 | 1.24E−05 | 0.0161 | PSEC0021 fis |
| 2 | A_23_P216966 | PTGS1 | 0.000455 | 0.78 | 2.95E−05 | 0.00703 | prostaglandin-endoperoxide |
| | | | | | | | synthase 1 |
| 2 | A_32_P17743 | | 0.000470 | 0.79 | 0.000174 | 0.00127 | |
| 2 | A_23_P79978 | SLC24A3 | 0.000581 | 0.78 | 4.70E−05 | 0.00718 | solute carrier family 24 member 3 |
| 2 | A_23_P43810 | LTBP1 | 0.000582 | 0.8 | 0.000185 | 0.00183 | latent transforming growth factor |
| | | | | | | | beta binding protein 1 |
| 2 | A_23_P6034 | TUBB1 | 0.000583 | 0.8 | 4.83E−05 | 0.00704 | tubulin, beta 1 |
| 2 | A_24_P176079 | WASF3 | 0.000620 | 0.76 | 0.00542 | 7.10E−05 | WAS protein family member 3 |
| 2 | A_23_P202823 | CTTN | 0.000655 | 0.76 | 0.00245 | 0.000175 | cortactin |
| 2 | A_23_P210358 | LIMS1 | 0.000690 | 0.79 | 0.000229 | 0.00208 | LIM and senescent cell antigen- |
| | | | | | | | like domains 1 |
| 2 | A_24_P929003 | ITGB3 | 0.000715 | 0.8 | 0.000133 | 0.00384 | integrin, beta 3 |
| 2 | A_23_P389118 | TMEM16F | 0.000766 | 0.72 | 1.73E−05 | 0.0339 | DKFZp313M0720 |
| 2 | A_23_P106042 | CKLFSF5 | 0.000769 | 0.78 | 0.000211 | 0.0028 | CKLF-like MARVEL |
| | | | | | | | transmembrane domain |
| | | | | | | | containing 5 |
| 2 | A_24_P160104 | TUBA8 | 0.000797 | 0.76 | 0.000232 | 0.00274 | tubulin, alpha 8 |
| 2 | A_23_P207507 | ABCC3 | 0.000809 | 0.77 | 0.000122 | 0.00536 | ATP-binding cassette, sub-family |
| | | | | | | | C member 3 |
| 2 | A_23_P102731 | SMOX | 0.000819 | 0.75 | 8.77E−05 | 0.00764 | spermine oxidase |
| 2 | A_32_P137604 | | 0.000838 | 0.81 | 0.000348 | 0.00202 | clone IMAGE: 3869276 |
| 2 | A_23_P104624 | KIAA0830 | 0.000910 | 0.75 | 0.000219 | 0.00378 | KIAA0830 protein, partial cds |
| 2 | A_23_P359277 | | 0.000965 | 0.76 | 6.65E−05 | 0.014 | ELOVL family member 7 |
| 2 | A_23_P151133 | NET-5 | 0.001003 | 0.75 | 0.000115 | 0.00875 | tetraspanin 9 |
| 2 | A_23_P105957 | ACTN1 | 0.001008 | 0.78 | 0.000899 | 0.00113 | actinin, alpha 1 |
| 2 | A_23_P17095 | TFPI | 0.001031 | 0.72 | 0.00136 | 0.000782 | tissue factor pathway inhibitor |
| 2 | A_23_P25974 | TTC7B | 0.001071 | 0.81 | 0.000634 | 0.00181 | tetratricopeptide repeat domain |
| | | | | | | | 7B |
| 2 | A_32_P168342 | C6orf25 | 0.001113 | 0.78 | 0.00016 | 0.00774 | FLJ35073 fis |
| 2 | A_23_P215913 | CLU | 0.001147 | 0.8 | 0.000129 | 0.0102 | clusterin |
| 2 | A_23_P416581 | GNAZ | 0.001155 | 0.8 | 0.000335 | 0.00398 | guanine nucleotide binding |
| | | | | | | | protein |
| 2 | A_24_P122337 | SYTL4 | 0.001175 | 0.74 | 0.000107 | 0.0129 | synaptotagmin-like 4 |
| 2 | A_23_P166633 | ITGB5 | 0.001207 | 0.79 | 0.000729 | 0.002 | integrin, beta 5 |
| 2 | A_24_P185186 | LOC201191 | 0.001257 | 0.71 | 2.60E−05 | 0.0608 | sterile alpha motif domain |
| | | | | | | | containing 14 |
| 2 | A_24_P333372 | | 0.001295 | 0.72 | 0.000492 | 0.00341 | FLJ35984 fis |
| 2 | A_23_P217998 | JAM3 | 0.001310 | 0.73 | 5.66E−05 | 0.0303 | junctional adhesion molecule 3 |
| 2 | A_23_P81930 | C6orf25 | 0.001357 | 0.61 | 7.97E−06 | 0.231 | chromosome 6 ORF 25 |
| 2 | A_23_P152160 | SNN | 0.001428 | 0.75 | 0.000294 | 0.00694 | stannin |
| 2 | A_23_P109974 | RAB6B | 0.001475 | 0.71 | 0.0035 | 0.000622 | RAB6B |
| 2 | A_23_P45524 | NGFRAP1 | 0.001761 | 0.75 | 0.000886 | 0.0035 | nerve growth factor receptor |
| | | | | | | | associated protein 1 |
| 2 | A_23_P7642 | SPARC | 0.001769 | 0.74 | 0.00031 | 0.0101 | secreted protein, acidic, cysteine- |
| | | | | | | | rich |
| 2 | A_23_P73457 | RUFY1 | 0.001850 | 0.77 | 0.0021 | 0.00163 | RUN and FYVE domain |
| | | | | | | | containing 1 |
| 2 | A_32_P136450 | | 0.001905 | 0.58 | 2.20E−05 | 0.165 | AF220206 Nedd4 WW domain- |
| | | | | | | | binding protein 2 |
| 2 | A_23_P17724 | SEP5 | 0.002039 | 0.6 | 2.52E−05 | 0.165 | septin 5 |
| 2 | A_23_P42975 | PRKAR2B | 0.002098 | 0.76 | 0.000284 | 0.0155 | protein kinase, cAMP-dependent, |
| | | | | | | | regulatory, type II, beta |
| 2 | A_23_P19987 | IMP-3 | 0.002357 | 0.75 | 0.000157 | 0.0354 | IGF-II mRNA-binding protein 3 |
| 2 | A_32_P162250 | ARHGAP18 | 0.002510 | 0.75 | 0.00348 | 0.00181 | Rho GTPase activating protein 18 |
| 2 | A_24_P251534 | CTDSPL | 0.002766 | 0.78 | 0.00126 | 0.00607 | carboxy-terminal domain, RNA |
| | | | | | | | polymerase II, polypeptide A |
| 2 | A_23_P391586 | | 0.002833 | 0.73 | 0.000818 | 0.00981 | tropomyosin 1 transcript variant 3 |
| 2 | A_24_P319923 | MYLK | 0.003091 | 0.72 | 0.00281 | 0.0034 | myosin, light polypeptide kinase |
| 2 | A_24_P13190 | ESAM | 0.003222 | 0.72 | 0.00145 | 0.00716 | endothelial cell adhesion |
| | | | | | | | molecule |
| 2 | A_23_P105562 | VWF | 0.003247 | 0.68 | 0.000172 | 0.0613 | von Willebrand factor |
| 2 | A_23_P111701 | GNG11 | 0.003249 | 0.67 | 0.00536 | 0.00197 | guanine nucleotide binding |
| | | | | | | | protein, gamma 11 |
| 2 | A_24_P254850 | KIAA0420 | 0.003707 | 0.74 | 0.000387 | 0.0355 | KIAA0420 mRNA |
| 2 | A_24_P79403 | PF4 | 0.003837 | 0.69 | 0.00162 | 0.00909 | platelet factor 4 |
| 2 | A_23_P121596 | PPBP | 0.004165 | 0.68 | 0.00064 | 0.0271 | pro-platelet basic protein |
| 2 | A_23_P143817 | MYLK | 0.004643 | 0.7 | 0.0055 | 0.00392 | myosin, light polypeptide kinase |
| 2 | A_23_P217428 | ARHGAP6 | 0.004934 | 0.72 | 0.00412 | 0.00591 | Rho GTPase activating protein 6 |
| 2 | A_23_P146584 | MGC17337 | 0.005001 | 0.74 | 0.00421 | 0.00594 | chromosome 9 ORF 30 |
| 2 | A_23_P149992 | PDLIM1 | 0.005416 | 0.57 | 7.66E−05 | 0.383 | PDZ and LIM domain 1 |
| 2 | A_23_P500844 | PDE5A | 0.005427 | 0.67 | 0.000425 | 0.0693 | phosphodiesterase 5A, cGMP- |
| | | | | | | | specific |
| 2 | A_23_P99906 | HOMER2 | 0.006003 | 0.69 | 0.000615 | 0.0586 | homer homolog 2 |
| 2 | A_24_P921366 | CALD1 | 0.006314 | 0.68 | 0.000291 | 0.137 | caldesmon 1 |
| 2 | A_23_P125233 | CNN1 | 0.006368 | 0.63 | 0.000524 | 0.0774 | calponin 1, basic |
| 2 | A_23_P8906 | LRP12 | 0.006827 | 0.7 | 0.00295 | 0.0158 | low density lipoprotein-related |
| | | | | | | | protein 12 |
| 2 | A_32_P140139 | F13A1 | 0.007655 | 0.67 | 0.002 | 0.0293 | coagulation factor XIII, A1 |
| | | | | | | | polypeptide |
| 2 | A_23_P360804 | CPNE5 | 0.008140 | 0.6 | 0.00162 | 0.0409 | copine V |
| 2 | A_24_P188071 | TUBA6 | 0.012196 | 0.59 | 0.037 | 0.00402 | tubulin, alpha 6 |
| 2 | A_23_P137697 | SELP | 0.013649 | 0.63 | 0.00786 | 0.0237 | selectin P |
| 2 | A_24_P892612 | | 0.016210 | 0.64 | 0.00457 | 0.0575 | DKFZp313A137 |
| 2 | A_23_P48212 | CLEC1B | 0.020106 | 0.66 | 0.00607 | 0.0666 | C-type lectin domain family 1, |
| | | | | | | | member B |
| 2 | A_23_P58396 | PDGFC | 0.039597 | 0.59 | 0.0564 | 0.0278 | platelet derived growth factor C |
| 2 | A_23_P209527 | | 0.040457 | 0.56 | 0.0186 | 0.088 | A31642 villin |
| 2 | A_23_P168556 | STX1A | 0.050571 | 0.53 | 0.0444 | 0.0576 | syntaxin 1A |
| 2 | A_32_P18723 | DKFZp762C1112 | 0.051355 | 0.52 | 0.0298 | 0.0885 | FLJ38153 fis |
| 2 | A_23_P52207 | BAMBI | 0.057018 | 0.46 | 0.00791 | 0.411 | BMP and activin membrane- |
| | | | | | | | bound inhibitor homolog |
| 2 | A_23_P431388 | SPOCD1 | 0.068662 | 0.52 | 0.0941 | 0.0501 | SPOC domain containing 1 |
| 2 | A_23_P371266 | DNM3 | 0.072500 | 0.49 | 0.0688 | 0.0764 | dynamin 3 |
| 2 | A_32_P179138 | | 0.087964 | 0.43 | 0.186 | 0.0416 | clone IMAGE: 5302158 |
| 3 | A_23_P111267 | SH3BGRL2 | 0.000145 | 0.84 | 5.79E−05 | 0.000364 | SH3 domain binding glutamic |
| | | | | | | | acid-rich protein like 2 |
| 3 | A_23_P219045 | HIST1H3D | 0.000158 | 0.76 | 4.14E−06 | 0.00601 | histone 1, H3d |
| 3 | A_24_P315256 | | 0.000195 | 0.64 | 2.00E−07 | 0.19 | |
| 3 | A_23_P91423 | C20orf112 | 0.000206 | 0.77 | 2.21E−05 | 0.00192 | chromosome 20 ORF 112 |
| 3 | A_23_P149545 | HIST2H2BE | 0.000234 | 0.75 | 7.28E−06 | 0.00752 | histone 2, H2be |
| 3 | A_23_P84448 | TUBA4 | 0.000329 | 0.65 | 8.46E−07 | 0.128 | tubulin, alpha 4 |
| 3 | A_23_P405295 | LCE3C | 0.000333 | 0.8 | 8.47E−05 | 0.00131 | late cornified envelope 3C |
| 3 | A_23_P152909 | ALOX12 | 0.000375 | 0.79 | 0.000108 | 0.0013 | arachidonate 12-lipoxygenase |
| 3 | A_23_P210939 | ITGB4BP | 0.000474 | 0.76 | 0.00022 | 0.00102 | integrin beta 4 binding protein |
| 3 | A_23_P4944 | CALM3 | 0.000497 | 0.64 | 2.13E−06 | 0.116 | calmodulin 3 |
| 3 | A_32_P221799 | HIST1H2AM | 0.000511 | 0.79 | 0.000133 | 0.00196 | histone 1, H2am |
| 3 | A_23_P436138 | MAX | 0.000609 | 0.66 | 5.84E−06 | 0.0636 | MYC associated factor X |
| 3 | A_24_P180680 | LAPTM4B | 0.000732 | 0.72 | 2.30E−05 | 0.0233 | lysosomal associated protein |
| | | | | | | | transmembrane 4 beta |
| 3 | A_24_P753476 | LOC340508 | 0.000758 | 0.8 | 0.000111 | 0.00518 | LOC340508 |
| 3 | A_24_P65373 | ITGA2B | 0.000988 | 0.64 | 5.64E−06 | 0.173 | integrin, alpha 2b |
| 3 | A_24_P918032 | LOC339005 | 0.001007 | 0.74 | 9.75E−05 | 0.0104 | LOC339005 |
| 3 | A_23_P160546 | FLJ11280 | 0.001239 | 0.76 | 0.000919 | 0.00167 | family with sequence similarity |
| | | | | | | | 63, member A |
| 3 | A_23_P41280 | PAICS | 0.001436 | 0.72 | 0.00562 | 0.000367 | phosphoribosylaminoimidazole |
| | | | | | | | carboxylase |
| 3 | A_24_P258633 | TUBB3 | 0.001463 | 0.08 | 0.000723 | 0.00296 | tubulin, beta 3 |
| 3 | A_24_P308506 | CML2 | 0.001503 | 0.73 | 0.00753 | 0.0003 | putative N-acetyltransferase |
| | | | | | | | Camello 2 |
| 3 | A_23_P206212 | THBS1 | 0.001565 | 0.72 | 0.000123 | 0.0199 | thrombospondin 1 |
| 3 | A_24_P382637 | GTPBP5 | 0.001741 | 0.7 | 0.00549 | 0.000552 | GTP binding protein 5 |
| 3 | A_32_P3385 | | 0.001798 | 0.72 | 0.00358 | 0.000903 | CS0DI060YD22 |
| 3 | A_23_P156708 | TNXB | 0.001969 | 0.68 | 0.043 | 9.02E−05 | tenascin XB |
| 3 | A_23_P74138 | TAGLN2 | 0.002092 | 0.66 | 5.86E−05 | 0.0747 | transgelin 2 |
| 3 | A_23_P215735 | ST7 | 0.002094 | 0.6 | 1.72E−05 | 0.255 | suppression of tumorigenicity 7 |
| 3 | A_23_P113701 | PDGFA | 0.002236 | 0.74 | 0.000365 | 0.0137 | platelet-derived growth factor |
| 3 | A_23_P121564 | GUCY1B3 | 0.002640 | 0.62 | 2.63E−05 | 0.265 | guanylate cyclase 1, soluble, beta 3 |
| 3 | A_24_P189533 | KIAA0830 | 0.002796 | 0.72 | 0.000774 | 0.0101 | KIAA0830 |
| 3 | A_32_P89709 | | 0.002878 | 0.72 | 0.000991 | 0.00836 | tropomyosin 1 |
| 3 | A_23_P15647 | NLK | 0.003035 | 0.64 | 5.98E−05 | 0.154 | nemo-like kinase |
| 3 | A_23_P24616 | CSE-C | 0.003070 | 0.67 | 0.00016 | 0.0589 | sialic acid acetylesterase |
| 3 | A_23_P73239 | NCKAP1 | 0.003580 | 0.67 | 0.000543 | 0.0236 | NCK-associated protein 1 |
| 3 | A_23_P3946 | NT5M | 0.003733 | 0.48 | 2.66E−05 | 0.524 | 5′,3′-nucleotidase, mitochondrial |
| 3 | A_23_P19624 | BMP6 | 0.004005 | 0.69 | 0.00225 | 0.00713 | bone morphogenetic protein 6 |
| 3 | A_24_P926709 | | 0.004013 | 0.61 | 0.00189 | 0.00852 | |
| 3 | A_23_P90407 | CASP14 | 0.004129 | 0.7 | 0.0084 | 0.00203 | caspase 14 |
| 3 | A_23_P167096 | VEGFC | 0.004234 | 0.67 | 0.000199 | 0.0901 | vascular endothelial growth |
| | | | | | | | factor C |
| 3 | A_23_P421843 | LOC201191 | 0.004449 | 0.57 | 4.90E−05 | 0.404 | sterile alpha motif domain |
| | | | | | | | containing 14 |
| 3 | A_23_P501831 | C5orf4 | 0.004848 | 0.65 | 0.000569 | 0.0413 | chromosome 5 ORF 4 |
| 3 | A_23_P417942 | FNBP1L | 0.005106 | 0.63 | 0.000349 | 0.0747 | formin binding protein 1-like |
| 3 | A_23_P307525 | ANKRD9 | 0.005136 | 0.52 | 6.28E−05 | 0.42 | ankyrin repeat domain 9 |
| 3 | A_32_P92212 | | 0.005401 | 0.62 | 0.000204 | 0.143 | IMAGE: 3271727 |
| 3 | A_23_P156284 | DBN1 | 0.005544 | 0.67 | 0.000456 | 0.0674 | drebrin 1 |
| 3 | A_23_P18539 | MMRN1 | 0.005781 | 0.57 | 0.000205 | 0.163 | multimerin 1 |
| 3 | A_24_P38387 | NDRG1 | 0.005842 | −0.6 | 0.00555 | 0.00615 | N-myc downstream regulated |
| | | | | | | | gene 1 |
| 3 | A_23_P155979 | EGF | 0.006397 | 0.67 | 0.00132 | 0.031 | epidermal growth factor (beta- |
| | | | | | | | urogastrone) |
| 3 | A_23_P401361 | PITPNM2 | 0.007348 | 0.62 | 0.00036 | 0.15 | phosphatidylinositol transfer |
| | | | | | | | protein, membrane-associated 2 |
| 3 | A_24_P385313 | PTPRF | 0.008340 | 0.64 | 0.00778 | 0.00894 | protein tyrosine phosphatase, |
| | | | | | | | receptor type, F |
| 3 | A_23_P141055 | TGFB1I1 | 0.008607 | 0.48 | 9.89E−05 | 0.749 | transforming growth factor beta 1 |
| | | | | | | | induced transcript 1 |
| 3 | A_24_P204257 | | 0.011918 | 0.65 | 0.00419 | 0.0339 | |
| 3 | A_23_P369899 | RIS1 | 0.013687 | 0.45 | 0.000409 | 0.458 | Ras-induced senescence 1 |
| 3 | A_24_P167654 | SLC8A3 | 0.016278 | 0.57 | 0.00207 | 0.128 | solute carrier family 8 member 3 |
| 3 | A_24_P405981 | | 0.018386 | 0.58 | 0.00313 | 0.108 | CS0DD001YH15 |
| 3 | A_23_P431853 | | 0.018431 | 0.6 | 0.0114 | 0.0298 | A-COL04217 |
| 3 | A_23_P367043 | MGC26484 | 0.020359 | 0.61 | 0.0132 | 0.0314 | CDC14 cell division cycle 14 |
| | | | | | | | homolog C |
| 3 | A_23_P135499 | CLIC4 | 0.026410 | 0.54 | 0.0094 | 0.0742 | chloride intracellular channel 4 |
| 3 | A_24_P32473 | | 0.026587 | 0.51 | 0.00198 | 0.357 | ELOVL family member 7 |
| 3 | A_23_P81934 | C6orf25 | 0.031498 | 0.53 | 0.00439 | 0.226 | chromosome 6 ORF 25 |
| 3 | A_24_P414999 | LAPTM4B | 0.032256 | 0.55 | 0.00439 | 0.237 | lysosomal associated protein |
| | | | | | | | transmembrane 4 beta |
| 3 | A_23_P207414 | MGC2744 | 0.034782 | 0.36 | 0.00143 | 0.846 | alanyl-tRNA synthetase domain |
| | | | | | | | containing 1 |
| 3 | A_32_P141437 | | 0.039100 | 0.51 | 0.0104 | 0.147 | FKSG73 |
| 3 | A_32_P59262 | | 0.057092 | 0.52 | 0.0205 | 0.159 | IMAGE: 3104077 |
| 3 | A_23_P61945 | MITF | 0.061935 | 0.51 | 0.028 | 0.137 | microphthalmia-associated |
| | | | | | | | transcription factor |
| 3 | A_23_P16866 | VIL1 | 0.066077 | 0.48 | 0.114 | 0.0383 | villin 1 |
| 3 | A_23_P127642 | ARHGEF12 | 0.072900 | 0.48 | 0.0146 | 0.364 | Rho guanine nucleotide exchange |
| | | | | | | | factor 12 |
| 3 | A_24_P713185 | | 0.075192 | 0.46 | 0.0287 | 0.197 | IMAGE: 4271522 |
| 3 | A_23_P69573 | GUCY1A3 | 0.106153 | 0.38 | 0.572 | 0.0197 | guanylate cyclase 1, soluble, |
| | | | | | | | alpha 3 |
| 3 | A_23_P257871 | DAB2 | 0.137768 | 0.43 | 0.146 | 0.13 | disabled homolog 2, mitogen- |
| | | | | | | | responsive phosphoprotein |
| 3 | A_24_P331882 | KIAA1211 | 0.188615 | 0.35 | 0.0604 | 0.589 | DKFZp434F117 |
| 3 | A_23_P154526 | GRB14 | 0.205232 | 0.32 | 0.432 | 0.0975 | growth factor receptor-bound |
| | | | | | | | protein 14 |
| 3 | A_23_P45304 | XK | 0.275935 | 0.2 | 0.162 | 0.47 | Kell blood group precursor |
| 3 | A_23_P104493 | PAPSS2 | 0.588394 | 0.12 | 0.349 | 0.992 | 3′-phosphoadenosine 5′- |
| | | | | | | | phosphosulfate synthase 2 |
| 4 | A_24_P143440 | DNCL2A | 0.000004 | 0.85 | 2.56E−08 | 0.000546 | dynein, light chain, roadblock- |
| | | | | | | | type 1 |
| 4 | A_23_P208788 | C19orf33 | 0.000040 | 0.85 | 0.000182 | 8.84E−06 | chromosome 19 ORF 33 |
| 4 | A_24_P68631 | HIST2H2AB | 0.000048 | 0.83 | 1.11E−05 | 0.000204 | histone 2, H2ab |
| 4 | A_23_P120364 | C20orf149 | 0.000060 | 0.8 | 9.45E−07 | 0.00385 | chromosome 20 ORF 149 |
| 4 | A_23_P202029 | SPFH1 | 0.000105 | 0.81 | 0.000107 | 0.000104 | SPFH domain family, member 1 |
| 4 | A_24_P287075 | MAP4K2 | 0.000111 | 0.8 | 4.13E−06 | 0.00296 | mitogen-activated protein kinase |
| | | | | | | | kinase kinase kinase 2 |
| 4 | A_23_P149301 | HIST3H2A | 0.000111 | 0.83 | 1.06E−05 | 0.00117 | histone 3, H2a |
| 4 | A_24_P6921 | | 0.000288 | 0.8 | 0.000193 | 0.00043 | LOC541471 protein |
| 4 | A_24_P135801 | | 0.000314 | 0.8 | 7.96E−05 | 0.00124 | CS0DF024YI14 |
| 4 | A_24_P45767 | FLJ21839 | 0.000417 | 0.8 | 0.00074 | 0.000235 | FLJ21839 |
| 4 | A_23_P42375 | RAB32 | 0.000526 | 0.76 | 9.01E−05 | 0.00307 | RAB32 |
| 4 | A_23_P354705 | ST8SIA1 | 0.000559 | −0.66 | 0.000185 | 0.00169 | ST8 alpha-N-acetyl-neuraminide |
| | | | | | | | alpha-2,8-sialyltransferase 1 |
| 4 | A_23_P407565 | CX3CR1 | 0.000593 | −0.64 | 0.00883 | 3.98E−05 | chemokine receptor 1 |
| 4 | A_23_P59045 | HIST1H2AE | 0.000607 | 0.77 | 0.000123 | 0.003 | histone 1, H2ae |
| 4 | A_24_P911960 | | 0.000642 | 0.77 | 7.30E−05 | 0.00565 | IMAGE: 1699732 |
| 4 | A_32_P184937 | | 0.000655 | 0.77 | 0.000585 | 0.000733 | BU678941 |
| 4 | A_23_P138117 | CAMTA1 | 0.000678 | 0.76 | 0.000183 | 0.00251 | calmodulin binding transcription |
| | | | | | | | activator 1 |
| 4 | A_32_P3113 | 2-Mar | 0.000802 | 0.79 | 0.000119 | 0.0054 | membrane-associated ring finger |
| | | | | | | | (C3HC4) 2 |
| 4 | A_32_P54137 | UQCRH | 0.001063 | 0.74 | 0.00576 | 0.000196 | ubiquinol-cytochrome c reductase |
| | | | | | | | hinge protein |
| 4 | A_23_P396626 | AP1GBP1 | 0.001214 | −0.66 | 0.00201 | 0.000733 | AP1 gamma subunit binding |
| | | | | | | | protein 1 |
| 4 | A_24_P227927 | IL21R | 0.001223 | −0.62 | 0.00571 | 0.000262 | interleukin 21 receptor |
| 4 | A_23_P110167 | MGST2 | 0.001371 | 0.78 | 0.00108 | 0.00174 | microsomal glutathione S- |
| | | | | | | | transferase 2 |
| 4 | A_23_P120933 | ATF4 | 0.001416 | 0.72 | 0.00105 | 0.00191 | activating transcription factor 4 |
| 4 | A_23_P55706 | RELB | 0.001465 | −0.64 | 0.00511 | 0.00042 | v-rel reticuloendotheliosis viral |
| | | | | | | | oncogene homolog B |
| 4 | A_24_P323835 | H3F3A | 0.001573 | 0.71 | 0.00029 | 0.00853 | H3 histone, family 3A |
| 4 | A_24_P273143 | MGC4677 | 0.001656 | 0.74 | 0.00294 | 0.000933 | hypothetical protein MGC4677 |
| 4 | A_23_P111037 | HIST1H3A | 0.001703 | 0.74 | 0.00107 | 0.00271 | histone 1, H3a |
| 4 | A_24_P223384 | HIST1H2AB | 0.001843 | 0.72 | 0.000724 | 0.00469 | histone 1, H2ab |
| 4 | A_23_P132285 | | 0.001983 | 0.74 | 0.00257 | 0.00153 | mercaptopyruvate |
| | | | | | | | sulfurtransferase |
| 4 | A_23_P52101 | NQO3A2 | 0.002328 | 0.72 | 0.00417 | 0.0013 | cytochrome b5 reductase 1 |
| 4 | A_24_P608790 | | 0.002619 | 0.7 | 0.00381 | 0.0018 | |
| 4 | A_24_P122732 | SLC41A1 | 0.002673 | −0.63 | 0.000901 | 0.00793 | solute carrier family 41, member 1 |
| 4 | A_32_P132317 | | 0.003079 | −0.63 | 0.00139 | 0.00682 | |
| 4 | A_23_P218817 | CPT1B | 0.003379 | −0.63 | 0.0066 | 0.00173 | carnitine palmitoyltransferase 1B |
| 4 | A_32_P132169 | | 0.003419 | 0.7 | 0.00191 | 0.00612 | |
| 4 | A_24_P102769 | UQCRH | 0.003712 | 0.69 | 0.00501 | 0.00275 | ubiquinol-cytochrome c reductase |
| | | | | | | | hinge protein |
| 4 | A_23_P148410 | FTHL17 | 0.005296 | 0.51 | 6.36E−05 | 0.441 | ferritin, heavy polypeptide-like |
| | | | | | | | 17 |
| 4 | A_23_P214330 | SERPINB1 | 0.005642 | 0.64 | 0.00584 | 0.00545 | serpin peptidase inhibitor, clade |
| | | | | | | | B, member 1 |
| 4 | A_32_P94521 | | 0.005688 | 0.68 | 0.000795 | 0.0407 | |
| 4 | A_32_P59302 | | 0.006040 | 0.61 | 0.00519 | 0.00703 | IMAGE: 6254031 |
| 4 | A_23_P121082 | GBE1 | 0.006706 | 0.67 | 0.0048 | 0.00937 | glucan branching enzyme 1 |
| 4 | A_24_P7934 | | 0.007472 | 0.62 | 0.00871 | 0.00641 | LOC391769 |
| 4 | A_23_P250671 | GPX1 | 0.044571 | 0.46 | 0.0413 | 0.0481 | glutathione peroxidase 1 |
| 4 | A_23_P69218 | LOC55831 | 0.047663 | 0.48 | 0.0292 | 0.0778 | transmembrane protein 111 |
| 4 | A_24_P913629 | | 0.083624 | 0.43 | 0.0222 | 0.315 | |
| 5 | A_23_P122007 | LOC90355 | 0.000013 | 0.78 | 5.58E−07 | 0.000291 | hypothetical gene supported by |
| | | | | | | | AF038182 |
| 5 | A_23_P210060 | MGC13057 | 0.000032 | 0.88 | 2.36E−06 | 0.000436 | DKFZp686I15210 |
| 5 | A_23_P138417 | RSU1 | 0.000035 | 0.8 | 1.88E−07 | 0.0066 | Ras suppressor protein 1 |
| 5 | A_23_P350591 | CXorf20 | 0.000038 | 0.83 | 7.91E−07 | 0.00181 | chromosome X ORF 20 |
| 5 | A_23_P114275 | PGRMC1 | 0.000052 | 0.75 | 2.08E−07 | 0.0131 | progesterone receptor membrane |
| | | | | | | | component 1 |
| 5 | A_24_P362540 | DDEF2 | 0.000137 | 0.84 | 1.92E−05 | 0.000971 | development and differentiation |
| | | | | | | | enhancing factor 2 |
| 5 | A_23_P167983 | HIST1H2AC | 0.000193 | 0.78 | 5.63E−06 | 0.0066 | histone 1, H2ac |
| 5 | A_23_P103070 | YWHAH | 0.000223 | 0.8 | 9.09E−06 | 0.00546 | tyrosine 3-monooxygenase |
| 5 | A_24_P273666 | GNAS | 0.000238 | 0.74 | 4.23E−06 | 0.0134 | GNAS complex locus |
| 5 | A_23_P333484 | HIST1H3H | 0.000275 | 0.79 | 0.000139 | 0.000545 | histone 1, H3h |
| 5 | A_23_P407203 | | 0.000286 | 0.66 | 4.61E−07 | 0.177 | FLJ42816 fis |
| 5 | A_23_P414273 | NID67 | 0.000314 | 0.82 | 0.000841 | 0.000117 | MSTP150 |
| 5 | A_23_P102391 | SLC40A1 | 0.000325 | 0.76 | 1.69E−05 | 0.00624 | solute carrier family 40 member 1 |
| 5 | A_23_P206018 | | 0.000403 | 0.76 | 1.69E−05 | 0.00963 | tropomyosin 1 |
| 5 | A_23_P72668 | SDPR | 0.000567 | 0.78 | 8.47E−05 | 0.00379 | serum deprivation response |
| 5 | A_24_P228550 | TUBB1 | 0.000606 | 0.82 | 0.000181 | 0.00203 | tubulin, beta 1 |
| 5 | A_23_P107612 | RAB27B | 0.000744 | 0.8 | 0.000151 | 0.00367 | RAB27B |
| 5 | A_23_P77145 | RAB11A | 0.000893 | 0.76 | 0.000114 | 0.007 | RAB11A |
| 5 | A_23_P502797 | WDFY1 | 0.000895 | −0.01 | 0.00393 | 0.000204 | WD repeat and FYVE domain |
| | | | | | | | containing 1 |
| 5 | A_23_P211910 | PLOD2 | 0.000943 | 0.77 | 0.000532 | 0.00167 | procollagen-lysine, 2- |
| | | | | | | | oxoglutarate 5-dioxygenase 2 |
| 5 | A_24_P44462 | TPM1 | 0.000991 | 0.73 | 4.86E−05 | 0.0202 | tropomyosin 1 |
| 5 | A_32_P125917 | | 0.001068 | 0.79 | 0.000144 | 0.00792 | BF238843 |
| 5 | A_23_P157128 | SCAP2 | 0.001259 | 0.72 | 0.00013 | 0.0122 | src family associated |
| | | | | | | | phosphoprotein 2 |
| 5 | A_32_P168349 | C6orf25 | 0.001322 | 0.74 | 9.06E−05 | 0.0193 | FLJ35073 fis |
| 5 | A_23_P216679 | CDC14B | 0.001372 | 0.66 | 1.61E−05 | 0.117 | CDC14 cell division cycle 14 |
| | | | | | | | homolog B |
| 5 | A_23_P63371 | TAL1 | 0.001478 | 0.8 | 0.00208 | 0.00105 | T-cell acute lymphocytic |
| | | | | | | | leukemia 1 |
| 5 | A_23_P12884 | GRK5 | 0.001545 | 0.73 | 6.47E−05 | 0.0369 | G protein-coupled receptor kinase 5 |
| 5 | A_23_P126836 | TNFSF4 | 0.001611 | 0.78 | 0.000238 | 0.0109 | tumor necrosis factor |
| | | | | | | | superfamily, member 4 |
| 5 | A_23_P23221 | GADD45A | 0.002036 | 0.7 | 0.000982 | 0.00422 | growth arrest and DNA-damage- |
| | | | | | | | inducible, alpha |
| 5 | A_23_P115608 | ARHGAP21 | 0.002171 | 0.72 | 0.000172 | 0.0274 | Rho GTPase activating protein 21 |
| 5 | A_24_P135444 | AMFR | 0.002179 | 0.67 | 0.000161 | 0.0295 | autocrine motility factor receptor |
| 5 | A_24_P118376 | UNQ9366 | 0.002790 | 0.61 | 3.09E−05 | 0.252 | carcinoembryonic antigen-related |
| | | | | | | | cell adhesion molecule 20 |
| 5 | A_23_P124476 | CLCN3 | 0.003450 | 0.63 | 0.000154 | 0.0773 | chloride channel 3 |
| 5 | A_32_P35751 | | 0.003593 | 0.69 | 0.000264 | 0.0489 | |
| 5 | A_32_P103558 | | 0.003653 | 0.74 | 0.00498 | 0.00268 | FLJ37480 fis |
| 5 | A_23_P334123 | CDA08 | 0.004356 | 0.58 | 7.53E−05 | 0.252 | T-cell immunomodulatory protein |
| 5 | A_23_P143902 | P2RY12 | 0.005046 | 0.69 | 0.00038 | 0.067 | purinergic receptor P2Y |
| 5 | A_23_P136693 | | 0.005253 | 0.68 | 0.0219 | 0.00126 | DKFZp686D0521 |
| 5 | A_23_P33947 | EFHC2 | 0.007107 | 0.66 | 0.0173 | 0.00292 | EF-hand domain containing 2 |
| 5 | A_23_P139486 | CDK2AP1 | 0.007218 | 0.62 | 0.000549 | 0.0949 | CDK2-associated protein 1 |
| 5 | A_23_P217611 | ARMCX3 | 0.007295 | 0.67 | 0.000649 | 0.082 | armadillo repeat containing, X- |
| | | | | | | | linked 3 |
| 5 | A_23_P86424 | NCOA4 | 0.007891 | 0.52 | 0.000265 | 0.235 | nuclear receptor coactivator 4 |
| 5 | A_23_P115375 | H3/o | 0.007956 | 0.62 | 0.03 | 0.00211 | histone H3/o |
| 5 | A_23_P91900 | SMC4L1 | 0.008477 | 0.04 | 0.00771 | 0.00932 | SMC4 structural maintenance of |
| | | | | | | | chromosomes 4-like 1 |
| 5 | A_23_P422083 | DKFZp762O076 | 0.009500 | 0.6 | 0.000586 | 0.154 | transmembrane protein 55A |
| 5 | A_23_P69226 | LOC55831 | 0.009851 | 0.64 | 0.00239 | 0.0406 | transmembrane protein 111 |
| 5 | A_23_P59547 | NT5C3 | 0.010860 | 0.62 | 0.00819 | 0.0144 | 5′-nucleotidase, cytosolic III |
| 5 | A_24_P500621 | | 0.012377 | 0.65 | 0.00382 | 0.0401 | FLJ23711 fis |
| 5 | A_24_P26897 | INPP5A | 0.012755 | 0.62 | 0.0033 | 0.0493 | inositol polyphosphate-5- |
| | | | | | | | phosphatase |
| 5 | A_23_P11025 | ZNF185 | 0.013107 | 0.64 | 0.000872 | 0.197 | zinc finger protein 185 |
| 5 | A_24_P349560 | EIF4E | 0.014011 | 0.54 | 0.000518 | 0.379 | eukaryotic translation initiation |
| | | | | | | | factor 4E |
| 5 | A_24_P941699 | PCGF5 | 0.015716 | 0.56 | 0.00233 | 0.106 | polycomb group ring finger 5 |
| 5 | A_24_P147927 | EFHC2 | 0.016470 | 0.54 | 0.00508 | 0.0534 | EF-hand domain containing 2 |
| 5 | A_23_P8763 | PTPN12 | 0.022823 | 0.6 | 0.00383 | 0.136 | protein tyrosine phosphatase, |
| | | | | | | | non-receptor type 12 |
| 5 | A_24_P81947 | CORO1C | 0.024231 | 0.58 | 0.00656 | 0.0895 | coronin, actin binding protein, 1C |
| 5 | A_23_P371239 | CMIP | 0.024670 | 0.46 | 0.0017 | 0.358 | c-Maf-inducing protein |
| 5 | A_23_P135494 | CLIC4 | 0.027543 | 0.58 | 0.00875 | 0.0867 | chloride intracellular channel 4 |
| 5 | A_23_P72643 | ADAM9 | 0.029899 | 0.54 | 0.00634 | 0.141 | metallopeptidase domain 9 |
| 5 | A_24_P503866 | | 0.049292 | 0.42 | 0.00968 | 0.251 | CS0DL005YE02 |
| 5 | A_24_P23411 | ARMCX3 | 0.052866 | 0.52 | 0.0137 | 0.204 | armadillo repeat containing, X- |
| | | | | | | | linked 3 |
| 5 | A_24_P633902 | ZNF364 | 0.054895 | 0.52 | 0.0123 | 0.245 | zinc finger protein 364 |
| 5 | A_32_P96134 | KIAA0877 | 0.065465 | 0.44 | 0.00698 | 0.614 | KIAA0877 |
| 5 | A_23_P201376 | SSX2IP | 0.071789 | 0.44 | 0.00901 | 0.572 | synovial sarcoma, X breakpoint 2 |
| | | | | | | | interacting protein |
| 5 | A_32_P6172 | | 0.075908 | 0.46 | 0.215 | 0.0268 | IMAGE: 5286843 |
| 5 | A_24_P27373 | PLDN | 0.101739 | 0.42 | 0.0477 | 0.217 | pallidin homolog |
| 5 | A_23_P96041 | FLJ22679 | 0.112988 | 0.4 | 0.0202 | 0.632 | FLJ22679 |
| 5 | A_32_P39384 | | 0.161655 | 0.09 | 0.306 | 0.0854 | IMAGE: 4823416 |
| 6 | A_23_P145965 | TPST1 | 0.000010 | 0.9 | 3.05E−05 | 2.97E−06 | tyrosylprotein sulfotransferase 1 |
| 6 | A_23_P33723 | CD163 | 0.000077 | 0.8 | 5.09E−05 | 0.000116 | CD163 antigen |
| 6 | A_24_P38081 | FKBP5 | 0.000138 | 0.86 | 3.82E−05 | 0.000498 | FK506 binding protein 5 |
| 6 | A_23_P111206 | FKBP5 | 0.000228 | 0.83 | 2.71E−05 | 0.00191 | FK506 binding protein 5 |
| 6 | A_23_P121602 | SAP30 | 0.000244 | 0.8 | 0.000484 | 0.000123 | sin3-associated polypeptide |
| 6 | A_23_P328729 | KLHL8 | 0.000273 | 0.82 | 0.000111 | 0.000672 | kelch-like 8 |
| 6 | A_23_P104804 | ZBTB16 | 0.000607 | 0.79 | 0.00389 | 9.47E−05 | zinc finger and BTB domain |
| | | | | | | | containing 16 |
| 6 | A_23_P99442 | FLT3 | 0.000780 | 0.79 | 0.000111 | 0.00548 | fms-related tyrosine kinase 3 |
| 6 | A_32_P806841 | ARL4A | 0.001443 | 0.6 | 8.40E−05 | 0.0248 | ADP-ribosylation factor-like 4A |
| 6 | A_32_P223985 | LOC388752 | 0.001917 | 0.74 | 0.00157 | 0.00234 | LOC388752 |
| 6 | A_24_P32215 | | 0.002288 | 0.57 | 7.92E−05 | 0.0661 | ADP-ribosylation factor-like 4B |
| 6 | A_23_P145761 | ARL4A | 0.002289 | 0.55 | 0.000139 | 0.0377 | ADP-ribosylation factor-like 4A |
| 6 | A_23_P53838 | IRS2 | 0.002515 | 0.74 | 0.00111 | 0.0057 | insulin receptor substrate 2 |
| 6 | A_24_P213296 | dJ341D10.1 | 0.003441 | 0.75 | 0.00764 | 0.00155 | dJ341D10.1 |
| 6 | A_23_P415401 | KLF9 | 0.050850 | 0.46 | 0.0117 | 0.221 | Kruppel-like factor 9 |
| 7 | A_23_P113212 | TMEM45A | 0.000027 | 0.82 | 4.53E−06 | 0.000165 | transmembrane protein 45A |
| 7 | A_32_P114020 | | 0.000060 | 0.85 | 8.32E−05 | 4.26E−05 | T32824 |
| 7 | A_32_P29140 | | 0.000061 | 0.85 | 0.00182 | 2.04E−06 | AA344632 |
| 7 | A_32_P130968 | | 0.000137 | 0.8 | 4.08E−05 | 0.000461 | IMAGE: 4826240 |
| 7 | A_23_P57658 | HRASLS | 0.000175 | 0.74 | 1.43E−06 | 0.0214 | HRAS-like suppressor |
| 7 | A_23_P381714 | CA13 | 0.000259 | 0.77 | 8.82E−05 | 0.000762 | carbonic anhydrase XIII |
| 7 | A_32_P131449 | | 0.000287 | 0.74 | 3.48E−06 | 0.0237 | |
| 7 | A_23_P151662 | MAX | 0.000310 | 0.81 | 3.69E−05 | 0.00261 | MYC associated factor X |
| 7 | A_23_P17130 | MGC13057 | 0.000319 | 0.8 | 7.85E−05 | 0.0013 | hypothetical protein MGC13057 |
| 7 | A_24_P76675 | MFAP3L | 0.000336 | 0.84 | 7.72E−05 | 0.00146 | microfibrillar-associated protein |
| | | | | | | | 3-like |
| 7 | A_23_P331253 | XPNPEP1 | 0.000479 | 0.76 | 2.30E−05 | 0.00998 | X-prolyl aminopeptidase 1 |
| 7 | A_24_P394510 | HIST1H2AJ | 0.000497 | 0.72 | 1.75E−05 | 0.0141 | histone 1, H2aj |
| 7 | A_23_P200001 | NEXN | 0.000587 | 0.76 | 0.000104 | 0.00331 | nexilin |
| 7 | A_32_P38745 | | 0.000645 | 0.8 | 0.000686 | 0.000606 | |
| 7 | A_24_P409971 | NEXN | 0.000784 | 0.78 | 5.04E−05 | 0.0122 | nexilin |
| 7 | A_24_P363615 | MTPN | 0.000858 | 0.72 | 0.000102 | 0.00721 | myotrophin |
| 7 | A_32_P196142 | | 0.000944 | 0.79 | 0.00224 | 0.000398 | |
| 7 | A_32_P808 | KIAA1458 | 0.000999 | 0.74 | 4.66E−05 | 0.0214 | KIAA1458 |
| 7 | A_32_P79041 | | 0.001349 | 0.68 | 0.0346 | 5.26E−05 | IMAGE: 6179261 |
| 7 | A_23_P217938 | SPHAR | 0.001657 | 0.73 | 0.00028 | 0.0098 | S-phase response |
| 7 | A_23_P132619 | OXTR | 0.001859 | 0.73 | 0.000898 | 0.00385 | oxytocin receptor |
| 7 | A_24_P453819 | | 0.002084 | 0.71 | 0.00118 | 0.00368 | IMAGE: 30330955 |
| 7 | A_23_P363344 | TPM1 | 0.002346 | 0.66 | 0.000173 | 0.0318 | tropomyosin 1 |
| 7 | A_23_P365685 | LIMS3 | 0.002380 | 0.77 | 0.000759 | 0.00746 | LIM and senescent cell antigen- |
| | | | | | | | like domains 3 |
| 7 | A_24_P148094 | LEPROT | 0.002416 | 0.7 | 0.000111 | 0.0526 | leptin receptor overlapping |
| | | | | | | | transcript |
| 7 | A_23_P131825 | TNNC2 | 0.002568 | 0.74 | 0.00822 | 0.000802 | troponin C type 2 |
| 7 | A_23_P39202 | C19orf33 | 0.002879 | 0.7 | 0.00086 | 0.00964 | chromosome 19 ORF33 |
| 7 | A_23_P16733 | RALB | 0.003196 | 0.65 | 0.0619 | 0.000165 | v-ral simian leukemia viral |
| | | | | | | | oncogene homolog B |
| 7 | A_23_P160336 | LEFTY1 | 0.003738 | 0.74 | 0.00102 | 0.0137 | left-right determination factor 1 |
| 7 | A_32_P117908 | | 0.004157 | 0.64 | 0.000163 | 0.106 | |
| 7 | A_24_P514678 | | 0.004737 | 0.69 | 0.00291 | 0.00771 | |
| 7 | A_23_P1126 | LEPROT | 0.004784 | 0.66 | 0.0021 | 0.0109 | leptin receptor overlapping |
| | | | | | | | transcript |
| 7 | A_23_P160582 | HT036 | 0.005785 | 0.64 | 0.00651 | 0.00514 | hydroxypyruvate isomerase |
| | | | | | | | homolog |
| 7 | A_32_P27878 | | 0.006556 | 0.67 | 0.00307 | 0.014 | AA399656 |
| 7 | A_23_P93282 | HIST1H3J | 0.007206 | 0.65 | 0.00775 | 0.0067 | histone 1, H3j |
| 7 | A_24_P570806 | | 0.008430 | 0.63 | 0.00345 | 0.0206 | IMAGE: 4814437 |
| 7 | A_32_P80532 | | 0.008928 | 0.48 | 9.84E−05 | 0.81 | BF733908 |
| 7 | A_24_P35478 | PARD3 | 0.012240 | 0.6 | 0.00165 | 0.0908 | par-3 partitioning defective 3 |
| | | | | | | | homolog |
| 7 | A_23_P38876 | LIPE | 0.012455 | 0.32 | 0.768 | 0.000202 | lipase, hormone-sensitive |
| 7 | A_23_P89902 | RTN2 | 0.013112 | 0.5 | 0.00033 | 0.521 | reticulon 2 |
| 7 | A_24_P879895 | | 0.013737 | 0.62 | 0.00293 | 0.0644 | IMAGE: 3883659 |
| 7 | A_24_P231104 | LEPR | 0.015491 | 0.56 | 0.00142 | 0.169 | leptin receptor |
| 7 | A_24_P524262 | | 0.019790 | 0.58 | 0.0611 | 0.00641 | Q80YT0 |
| 7 | A_23_P38106 | SPHK1 | 0.023166 | 0.45 | 0.000905 | 0.593 | sphingosine kinase 1 |
| 7 | A_23_P137173 | TMSNB | 0.023397 | 0.53 | 0.00238 | 0.23 | thymosin-like 8 |
| 7 | A_32_P25639 | BET3L | 0.039122 | 0.51 | 0.198 | 0.00773 | FLJ11180 fis |
| 7 | A_23_P426663 | MITF | 0.044996 | 0.54 | 0.00703 | 0.288 | microphthalmia-associated |
| | | | | | | | transcription factor |
| 7 | A_23_P169756 | HIPK2 | 0.045615 | 0.39 | 0.549 | 0.00379 | homeodomain interacting protein |
| | | | | | | | kinase 2 |
| 7 | A_23_P92025 | CIDEC | 0.059978 | 0.34 | 0.00363 | 0.991 | cell death-inducing DFFA-like |
| | | | | | | | effector c |
| 7 | A_32_P181297 | | 0.061180 | 0.44 | 0.488 | 0.00767 | CS0DK012YG12 |
| 7 | A_23_P377214 | FLJ32384 | 0.065383 | 0.48 | 0.0217 | 0.197 | hexamthylene bis-acetamide |
| | | | | | | | inducible 2 |
| 7 | A_32_P4433 | | 0.070070 | 0.46 | 0.098 | 0.0501 | BU602485 |
| 7 | A_23_P213050 | HPGD | 0.089001 | 0.47 | 0.0483 | 0.164 | hydroxyprostaglandin |
| | | | | | | | dehydrogenase 15-(NAD) |
| 7 | A_23_P328740 | LOC93082 | 0.124378 | 0.42 | 0.0874 | 0.177 | BC012317 |
| 7 | A_24_P347447 | DAAM1 | 0.134365 | 0.39 | 0.177 | 0.102 | dishevelled associated activator |
| | | | | | | | of morphogenesis 1 |
| 7 | A_23_P54116 | DAAM1 | 0.154932 | 0.39 | 0.0934 | 0.257 | dishevelled associated activator |
| | | | | | | | of morphogenesis 1 |
| 7 | A_23_P65674 | TMOD3 | 0.257564 | 0.32 | 0.273 | 0.243 | tropomodulin 3 |
| 7 | A_32_P225135 | | 0.483072 | 0.23 | 0.259 | 0.901 | IMAGE: 5277859 |
| 8 | A_23_P46369 | RAB13 | 0.000031 | 0.83 | 8.86E−06 | 0.000106 | RAB13 |
| 8 | A_23_P130961 | ELA2 | 0.000132 | 0.84 | 1.56E−05 | 0.00111 | elastase 2 |
| 8 | A_23_P140384 | CTSG | 0.000271 | 0.82 | 7.50E−05 | 0.000982 | cathepsin G |
| 8 | A_23_P86653 | PRG1 | 0.000326 | 0.75 | 2.86E−05 | 0.00372 | proteoglycan 1, secretory granule |
| 8 | A_23_P141173 | MPO | 0.000634 | 0.78 | 6.51E−05 | 0.00618 | myeloperoxidase |
| 8 | A_23_P167005 | GPR160 | 0.001061 | 0.72 | 0.00938 | 0.00012 | G protein-coupled receptor 160 |
| 8 | A_23_P121716 | ANXA3 | 0.001315 | 0.72 | 0.00144 | 0.0012 | annexin A3 |
| 8 | A_23_P326080 | DEFA4 | 0.001467 | 0.7 | 0.000333 | 0.00646 | defensin, alpha 4, corticostatin |
| 8 | A_24_P347378 | ALOX5AP | 0.001541 | 0.71 | 0.00144 | 0.00165 | arachidonate 5-lipoxygenase- |
| | | | | | | | activating protein |
| 8 | A_23_P201193 | TSPAN2 | 0.001921 | 0.71 | 0.000489 | 0.00755 | tetraspanin 2 |
| 8 | A_23_P150903 | MLSTD1 | 0.001962 | 0.71 | 0.00104 | 0.0037 | male sterility domain containing 1 |
| 8 | A_23_P131789 | BPI | 0.002874 | 0.66 | 0.00113 | 0.00731 | bactericidal/permeability- |
| | | | | | | | increasing protein |
| 8 | A_23_P169437 | LCN2 | 0.002906 | 0.67 | 0.00364 | 0.00232 | lipocalin 2 |
| 8 | A_23_P159952 | BEX1 | 0.003754 | 0.66 | 0.0052 | 0.00271 | brain expressed, X-linked 1 |
| 8 | A_23_P69171 | SUCNR1 | 0.004568 | 0.65 | 0.00346 | 0.00603 | succinate receptor 1 |
| 8 | A_23_P71981 | ERAL1 | 0.009085 | 0.62 | 0.00907 | 0.0091 | Era G-protein-like 1 |
| 9 | A_24_P63019 | IL1R2 | 0.000002 | 0.83 | 2.00E−06 | 3.01E−06 | interleukin 1 receptor, type II |
| 9 | A_23_P60627 | ALOX15B | 0.000010 | 0.85 | 0.000125 | 8.69E−07 | arachidonate 15-lipoxygenase, |
| | | | | | | | second type |
| 9 | A_23_P4036 | HT008 | 0.000015 | 0.89 | 1.98E−05 | 1.17E−05 | testis expressed sequence 2 |
| 9 | A_23_P117582 | JDP2 | 0.000034 | 0.84 | 0.000231 | 5.06E−06 | jun dimerization protein 2 |
| 9 | A_32_P224094 | ZNF143 | 0.000056 | 0.79 | 0.000529 | 5.91E−06 | zinc finger protein 143 |
| 9 | A_24_P202567 | ITPKC | 0.000062 | 0.84 | 1.65E−05 | 0.000232 | inositol 1,4,5-trisphosphate 3- |
| | | | | | | | kinase C |
| 9 | A_23_P162668 | CPM | 0.000082 | 0.8 | 0.000296 | 2.25E−05 | carboxypeptidase M |
| 9 | A_23_P255104 | LHFPL2 | 0.000101 | 0.79 | 2.49E−06 | 0.0041 | lipoma HMGIC fusion partner- |
| | | | | | | | like 2 |
| 9 | A_23_P155765 | HMGB2 | 0.000113 | 0.82 | 2.57E−05 | 0.000497 | high-mobility group box 2 |
| 9 | A_23_P169529 | HRB | 0.000139 | 0.8 | 5.28E−06 | 0.00365 | HIV-1 Rev binding protein |
| 9 | A_23_P116195 | | 0.000162 | 0.81 | 0.00481 | 5.45E−06 | Q7PKG0 |
| 9 | A_23_P11201 | GPR34 | 0.000167 | 0.84 | 4.19E−05 | 0.000666 | G protein-coupled receptor 34 |
| 9 | A_23_P388900 | SLC22A15 | 0.000210 | 0.81 | 0.000107 | 0.000414 | solute carrier family 22, member |
| | | | | | | | 15 |
| 9 | A_24_P938352 | CPM | 0.000269 | 0.81 | 0.000125 | 0.000577 | carboxypeptidase M |
| 9 | A_23_P423864 | PHC2 | 0.000402 | 0.76 | 6.02E−05 | 0.00268 | polyhomeotic-like 2 |
| 9 | A_23_P138725 | MARVELD1 | 0.000564 | 0.79 | 0.000175 | 0.00182 | MARVEL domain containing 1 |
| 9 | A_24_P269687 | TOR1A | 0.000586 | 0.7 | 5.25E−05 | 0.00653 | torsin family 1, member A |
| 9 | A_24_P913115 | | 0.000814 | 0.77 | 0.000137 | 0.00484 | CS0DK002YE20 |
| 9 | A_23_P93562 | SESN1 | 0.001184 | 0.74 | 0.000356 | 0.00394 | sestrin 1 |
| 9 | A_23_P104798 | IL18 | 0.001190 | 0.78 | 0.000343 | 0.00413 | interleukin 18 |
| 9 | A_23_P8640 | GPR30 | 0.001536 | 0.76 | 0.00068 | 0.00347 | G protein-coupled receptor 30 |
| 9 | A_24_P78531 | CLEC4E | 0.002161 | 0.77 | 0.00251 | 0.00186 | C-type lectin domain family 4, |
| | | | | | | | member E |
| 9 | A_23_P215566 | AHR | 0.002474 | 0.75 | 0.00358 | 0.00171 | aryl hydrocarbon receptor |
| 9 | A_23_P415021 | DKFZP586A0522 | 0.003217 | 0.71 | 0.00639 | 0.00162 | DKFZP586A0522 |
| 9 | A_24_P154037 | IRS2 | 0.003607 | 0.8 | 0.00215 | 0.00605 | insulin receptor substrate 2 |
| 9 | A_24_P750164 | LOC151438 | 0.004384 | 0.71 | 0.00223 | 0.00862 | \FLJ31315 fis |
| 9 | A_23_P98085 | PTEN | 0.004927 | 0.68 | 0.00274 | 0.00886 | phosphatase and tensin homolog |
| 9 | A_24_P233995 | FLJ22390 | 0.008645 | 0.69 | 0.0087 | 0.00859 | MOCO sulphurase C-terminal |
| | | | | | | | domain containing 1 |
| 10 | A_24_P235266 | GRB10 | 0.000044 | 0.8 | 2.84E−06 | 0.000697 | growth factor receptor-bound |
| | | | | | | | protein 10 |
| 10 | A_23_P122863 | GRB10 | 0.000207 | 0.76 | 5.88E−06 | 0.0073 | growth factor receptor-bound |
| | | | | | | | protein 10 |
| 10 | A_24_P360674 | CDKN2B | 0.002052 | 0.69 | 8.42E−05 | 0.05 | cyclin-dependent kinase inhibitor |
| | | | | | | | 2B |
| 10 | A_24_P323084 | FLJ39421 | 0.007251 | 0.68 | 0.00106 | 0.0496 | chromosome 17 ORF 55 |
| 10 | A_23_P502470 | IL6ST | 0.007330 | 0.67 | 0.00727 | 0.00739 | interleukin 6 signal transducer |

TABLE 2

| | All Days post-transplant | | | | ≦180 days post-transplant | | | |
|---|---|---|---|---|---|---|---|---|
| | | Mean | | | | Mean | | |
| Gene/ | Mean R | NR | Ratio | | Mean R | NR | Ratio | |
| Protein | (n = 39) | (n = 65) | R/NR | p-value* | (n = 28) | (n = 46) | R/NR | p-value* |
| | 27.4 | 23.9 | NA | 0.01 | 28.4 | 22.4 | NA | 0.0004 |
| IL1R2 | 34.3 | 33.6 | 0.62 | 0.009 | 34.4 | 33.2 | 0.44 | 0.0003 |
| PDCD1 | 32 | 32.4 | 1.32 | 0.03 | 32 | 32.4 | 1.32 | 0.06 |
| FLT3 | 32 | 31.6 | 0.76 | 0.11 | 32.2 | 31.5 | 0.62 | 0.02 |
| PF4 | 25 | 24.8 | 0.87 | 0.18 | 25 | 24.8 | 0.87 | 0.27 |
| ITGAM | 26.9 | 26.8 | 0.93 | 0.22 | 27 | 26.7 | 0.81 | 0.07 |
| SEMA7A | 34.3 | 34.4 | 1.07 | 0.31 | 34.3 | 34.5 | 1.15 | 0.16 |
| RHOU | 29.8 | 29.9 | 1.07 | 0.41 | 29.8 | 29.9 | 1.07 | 0.24 |
| G6b | 26.7 | 26.5 | 0.87 | 0.46 | 26.6 | 26.5 | 0.93 | 0.72 |
| ITGA4 | 27.6 | 27.6 | 1 | 0.47 | 27.6 | 27.7 | 1.07 | 0.31 |
| WDR40A | 28.9 | 28.8 | 0.93 | 0.68 | 28.7 | 28.8 | 1.07 | 0.88 |
| MIR | 29.4 | 29.3 | 0.93 | 0.82 | 29.3 | 29.3 | 1 | 0.85 |

*Significant values in larger red typeface

TABLE 3

| | |
|---|---|
| All times post | <180 da post |
| transplant | transplant |
| R (n = 38)/ | R (n = 27)/ |
| NR (n = 55) | NR (n = 40) |

| | Fold | | Fold | |
|---|---|---|---|---|
| Gene/Protein | Change | p-value* | Change | p-value |
| IL1R1 | 0.67 | 0.01 | 0.55 | 0.0008 |
| TSC22D3 | 0.8 | 0.01 | 0.72 | 0.0009 |
| FKBP5 | 0.85 | 0.18 | 0.68 | 0.007 |
| THBS1 | 0.73 | 0.04 | 0.68 | 0.03 |
| CD163 | 0.85 | 0.2 | 0.72 | 0.03 |
| ABCB1 | 1.1 | 0.41 | 1.28 | 0.07 |
| ANXA1 | 0.89 | 0.1 | 0.86 | 0.1 |
| IL1B | 1.29 | 0.19 | 1.45 | 0.11 |
| EPOR | 0.9 | 0.06 | 0.91 | 0.17 |
| DUSP1 | 0.88 | 0.39 | 0.79 | 0.21 |
| SGK | 1.08 | 0.5 | 1.16 | 0.27 |
| TGFB1 | 0.94 | 0.19 | 0.94 | 0.3 |
| IL7R | 1.08 | 0.54 | 1.19 | 0.3 |
| NFKBIA | 0.92 | 0.41 | 0.9 | 0.43 |
| NR3C1 | 1.01 | 0.76 | 1.02 | 0.52 |
| IL4R | 0.98 | 0.75 | 0.97 | 0.56 |
| SELP | 0.88 | 0.36 | 0.93 | 0.62 |
| IL1RN | 0.97 | 0.73 | 0.97 | 0.78 |
| THBS2 | 0.97 | 0.74 | 1.03 | 0.79 |
| ITGAX | 1.02 | 0.8 | 0.96 | 0.86 |
| TNFRSF1 | 0.94 | 0.61 | 1.02 | 0.89 |
| ADA | 1.26 | 0.002 | 1.35 | 0.0008 |
| GZMA | 1.19 | 0.15 | 1.4 | 0.01 |
| TRBC1 | 1.27 | 0.8 | 1.5 | 0.02 |
| FLT3LG | 1.16 | 0.12 | 1.31 | 0.03 |
| CD28 | 1.21 | 0.12 | 1.33 | 0.08 |
| CD8A | 1.15 | 0.37 | 1.32 | 0.1 |
| PDCD1L | 1.2 | 0.6 | 1.21 | 0.12 |
| CTLA4 | 1.19 | 0.17 | 1.23 | 0.18 |
| CD274 | 1.08 | 0.38 | 1.15 | 0.2 |
| CD4 | 1.01 | 0.87 | 1.08 | 0.35 |
| NFKB1 | 1.09 | 0.03 | 1.1 | 0.02 |
| TNF | 1.21 | 0.06 | 1.32 | 0.03 |

### EXAMPLES

### Example 1

### Study Objectives and Subjects

Nucleic acid technologies were used to produce gene expression profiles for PBMC samples from subjects who had been treated with various dosages of steroid and were enrolled in the Cardiac Allograft Rejection Gene Expression Observational (CARGO) and the Lung Allograft Rejection Gene Expression Observational (LARGO) studies. All studies were approved by local Institutional Review Boards.

The CARGO study was initiated in 2001 to study gene expression in blood samples as a means for managing transplant rejection in cardiac patients. The eight transplant centers contributing to the studies handle more than 20% of cardiac transplants. The LARGO study was initiated in 2004 to collect blood samples and clinical data, including the results from TBB from lung transplant subjects, at fourteen centers in five different countries.

Microarrays as described in Example 10 were used to study gene expression in 95 samples from LARGO subjects being treated with 5-40 mg of steroid, 68 samples from CARGO subjects being treated with 1-100 mg of steroid, and 56 samples from CARGO or LARGO subjects being treated with 0-50 mg of steroid for CMV infection.

RT-PCR was used in exemplary and pathways studies with PBMC samples from CARGO subjects between 30 days and 12 months post-transplant whose transplants were graded as rejection or non-rejection. The principle inclusion criteria were: a) clinically stable defined as absence of signs or symptoms of acute cardiac allograft rejection, b) histologically stable defined as current EMB indicating non-rejection, c) absence of cardiac dysfunction by invasive hemodynamics and/or echocardiogram, and d) absence of ISHLT (International Society for Heart and Lung Transplant)≧3A rejection, graft dysfunction, or administration of rejection therapy within 30 days prior to enrollment. The demographic and treatment characteristics of the cardiac transplant subjects are shown in the following Table 4.

TABLE 4

| | Subjects-all days post | | | Subjects ≦180 days post | | |
|---|---|---|---|---|---|---|
| | transplant | | | transplant | | |
| Groups-No Subjects | R = 39 | NR = 65 | p-value | R = 28 | NR = 46 | p-value |
| Median Age (Range) | 60 (25-68) | 59 (8-76) | 0.58 | 59 (25-68) | 59 (8-76) | 0.73 |
| Sex-Male (%) | 32 (82.1) | 54 (83.1) | 1 | 22 (78.6) | 41 (53.6) | 0.31 |
| Race-No (%) | | | 0.33 | | | 0.025 |
| White | 23 (59.0) | 47 (72.3) | | 15 (53.6) | 38 (82.6) | |
| Black | 10 (25.6) | 10 (15.6) | | 8 (28.6) | 5 (10.9) | |
| Other | 6 (15.4) | 8 (12.1) | | 5 (17.8) | 3 (6.5) | |
| Immunosuppression Regimen-No (%) | | | 0.32 | | | 0.29 |
| Cyclosporine/Mycophenolate | 20 (51.3) | 37 (56.9) | | 15 (53.6) | 28 (60.9) | |
| Cyclosporine/Sirolimus | 1 (2.6) | 2 (3.1) | | 1 (3.6) | 2 (4.3) | |
| Tacrolimus/Mycophenolate | 10 (25.6) | 19 (29.2) | | 6 (21.4) | 12 (26.1) | |
| Tacrolimus/Sirolimus | 6 (15.4) | 3 (4.6) | | 5 (17.9) | 2 (4.3) | |
| Other | 2 (5.1) | 4 (6.2) | | 1 (3.6) | 2 (4.3) | |
| Median Dose (Range) | | | | | | |
| Index Sample | 10 (2-30) | 10 (1-60) | 0.62 | 13.25 (2-30) | 12.5 (1-60) | 0.75 |
| R/NR Sample | 7.5 (1-25) | 7.5 (2-20) | 0.8 | 10 (2-25) | 10 (2.5-20) | 0.6 |
| Post-recovery Sample | 10 (1-80) | 6 (1-20) | 0.003 | 10 (2-80) | 7.5 (2-20) | 0.003 |
| Days Post-Transplant-Median (Range) | | | | | | |
| Index Sample | 138 (32-491) | 133 (33-317) | 0.3 | 93 (32-180) | 83 (33-177) | 0.54 |
| R/NR Sample | 180 (53-565) | 166 (56-342) | 0.33 | 130 (53-240) | 124 (56-242) | 0.58 |
| Post-recovery Sample | 189 (62-579) | 228 (70-471) | 0.56 | 155 (62-249) | 152 (70-304) | 0.35 |
| Days from Index to R/NR | 35 (14-77) | 34 (14-76) | 0.99 | 32 (14-63) | 31 (14-70) | 0.89 |
| ISHLT Biopsy-No (%) | | | 0.0006 | | | 0.008 |
| Grade 0 | 12 (30.8) | 43 (66.2) | | 9 (32.1) | 30 (65.2) | |
| Grade 1A | 27 (69.2) | 22 (34.4) | | 19 (67.9) | 16 (34.8) | |

Column 1 of the table characterizes the subjects, immunosuppression regimen, days post-transplant and ISHLT grades. Columns 2, 3 and 4 show the data for rejection (R) and non-rejection (NR) subjects and p-values for characteristics all days post-transplant. Columns 4, 5, and 6 show the data for rejection (R) and non-rejection (NR) subjects and p-values for characteristics <180 days post transplant.

Subjects in both the R and NR groups were on standard steroid weaning protocols with no significant difference (p=0.75) in steroid dose. A two-tailed independent t-test or a Fisher Exact test was used to compare quantitative characteristics, and a Wald (Mann Whitney) test was used to compare categorical characteristics. There was no significant difference in the distribution of characteristics between groups except that ISHLT 1A biopsies and African-Americans were more prevalent in the R group.

### Example 2

### Sample Collection, Transplant Protocol, and Immunosuppressive Therapy

A blood sample was collected from each subject at each clinical encounter, and clinical data including results of EMB or TBB, immunosuppressive regime, laboratory data, and clinical complications were obtained. Samples were processed as described in Example 8.

Standard cardiac transplant center protocols generally require invasive EMBs to be performed weekly in the first 30 days post transplant (4 biopsies), every two weeks between 31-90 days post transplant (4 biopsies), every 4 weeks between 91-180 days post transplant (3 biopsies), and every 8 weeks between 181-365 days post transplant (3 biopsies). Histology was graded by a local pathologist and two or three pathologists blinded to subject data and outcomes. Agreement of at least two of the pathologists was required to diagnose ISHLT≧3A rejection, and agreement of three pathologists was required for ISHLT 0/1A non-rejection.

Standard lung transplant center protocols generally require at least six invasive TBBs during the first six months post transplant. These tissue samples are examined by at least three pathologists for signs of rejection and rated on a five point ISHLT scale of increasing severity based on the extent of perivascular inflammation, A0=normal lung tissue, A1=minimal, A2=mild, A3=moderate, and A4=severe rejection. A TBB rated≧A2 generally requires therapeutic intervention.

All subjects received center-specific immunosuppressive therapy consisting of cyclosporine or tacrolimus in combination with either mycophenolate mofetil or sirolimus and corticosteroids. The cardiac rejection group (R) had 39 subjects who progressed to acute cellular rejection within 12 weeks. The control group (NR) had 65 subjects who remained rejection-free and were matched with subjects in the rejection group by demographic characteristics, time post-transplant, and immunosuppressive therapy.

### Example 3

### Steroid Modulated Nucleic Acids and Their Expression

Steroid modulated genes are described in the clusters of Table 1, in the diagnostic set of genes of Table 5, in the pathways genes of Table 3, and among the sequences listed in the published applications and patents incorporated by reference herein in their entirely and shown in the table below.

TABLE 5

| Title | Application No; Filing Date | Patent/Publication No |
|---|---|---|
| Methods And Compositions | USSN 10/131,827; Apr. 24, 2002 | USPN 6,905,827 |
| For Diagnosing And | PCT/US03/13015; Apr. 24, 2003 | WO03/090694 |
| Monitoring Autoimmune | | |
| And Chronic Inflammatory | | |
| Diseases | | |
| Methods And Compositions | USSN 10/325,899; | US2003/123086 |
| For Diagnosing And | Dec. 20, 2002 | WO04/042346 |
| Monitoring Transplant | PCT/US03/129456 | |
| Rejection | | |
| Leukocyte Expression | PCT/US01/47856; | WO02/057414 |
| Profiling | Oct. 22, 2001 | |

The steroid modulated genes were identified using at least one statistical method on nucleic acid expression from the microarray study as described in Example 4 and RT-PCR studies as described in Example 5. Primers and probe sets for use in a diagnostic set for detecting genes modulated by steroids can be generated as described in Examples 11 and 12.

### Example 4

### Microarray Study and Results

Protocols used with the microarrays are described in Examples 9 and 10. For the microarray studies, the manufacturer's software was used to download microarray data. To be included in the analysis, a probe had to be flagged as present (versus marginal or absent) and have a signal of at least 100 for at least 80% of the arrays.

Nucleic acids expressed on Human Genome CGH 44A microarrays (Agilent Technologies, Palo Alto Calif.) that correlated with steroid treatment were identified separately in the samples from the CARGO and LARGO projects. Feature Extraction and GeneSpring software (Agilent Technologies) were used to download microarray data. As shown in the first table in Example 1, the initial filtering flagged 28,997 out of 41,000 probes. Signals were normalized to the median expression of each chip to achieve chip-to-chip comparability.

K-means clustering was applied to the expression of 28,997 nucleic acids in 219 samples as shown in the table below. The parameters for clustering were the number of clusters (20), number of iterations (400), and similarity measure (p-value, Pearson correlation). In one alternative, similarity measure can be a t-test.

In the initial analysis, nucleic acid expression converged after 147 iterations. Using a p-value<0.01, CARGO samples showed expression in 3,604 genes; LARGO samples, in 699 genes. The CARGO and LARGO samples had 278 expressed nucleic acids in common, and cluster 14 (highlighted) was found to be highly enriched in steroid modulated (SM) genes (62.9%), with another 24.7% whose expression correlated with steroid dose (CSD).

TABLE 6

| Cluster | No. Genes | No. SM Genes | % SM Genes | % of CSD Genes |
|---|---|---|---|---|
| 1 | 1904 | 2 | 0.7 | 0.1 |
| 2 | 1562 | 2 | 0.7 | 0.1 |
| 3 | 2218 | 2 | 0.7 | 0.1 |
| 4 | 3236 | 2 | 0.7 | 0.1 |
| 5 | 2212 | 5 | 1.8 | 0.2 |
| 6 | 1305 | 1 | 0.4 | 0.1 |
| 7 | 2024 | 1 | 0.4 | 0 |
| 8 | 803 | 0 | 0 | 0 |
| 9 | 1174 | 2 | 0.7 | 0.2 |
| 10 | 2059 | 24 | 8.6 | 1.2 |
| 11 | 975 | 1 | 0.4 | 0.1 |
| 12 | 1219 | 2 | 0.7 | 0.2 |
| 13 | 336 | 0 | 0 | 0 |
| 14 | 709 | 175 | 62.9 | 24.7 |
| 15 | 304 | 20 | 7.2 | 6.6 |
| 16 | 1015 | 3 | 1.1 | 0.3 |
| 17 | 3303 | 6 | 2.2 | 0.2 |
| 18 | 515 | 3 | 1.1 | 0.6 |
| 19 | 981 | 0 | 0 | 0 |
| 20 | 1143 | 27 | 9.7 | 2.4 |
| Total | 28997 | 278 | 100 | 37.2 |

Column one of Table 6 shows the cluster number; column two, the number of genes in that cluster; column 3, the number of steroid modulated genes; column four, the percent of steroid modulated genes; and column five, the percent of genes correlated with steroid dose.

Candidate steroid modulated nucleic acids (709 genes from cluster 14 and 278 steroid dose correlated genes) were subjected to additional rounds of K-means clustering. The parameters were number of clusters (40), number of iterations (100), and similarity measure (p-value, Pearson correlation). After each round, any cluster containing zero or one steroid modulated nucleic acid was eliminated. Clusters containing two or more steroid modulated nucleic acids were combined for next round of clustering. After four rounds of K-means clustering, 518 genes were in clusters that contained two or more steroid modulated nucleic acids and 262 (50.5%) were nucleic acids whose expression were correlated with steroid dose (data not shown). These 518 genes were subjected to further rounds of clustering with the parameters: number of clusters (10), number of iterations (100), similarity measure (p-value, Pearson correlation). As shown in the table below, all genes had converged into ten clusters after 14 iterations. The 518 steroid modulated genes are described in their respective clusters in Table 1.

TABLE 7

| Cluster | No. of SM genes | No. CSD Genes |
|---|---|---|
| 1 | 116 | 46 |
| 2 | 95 | 55 |
| 3 | 73 | 21 |
| 4 | 45 | 40 |
| 5 | 67 | 20 |
| 6 | 15 | 11 |
| 7 | 58 | 22 |
| 8 | 16 | 16 |
| 9 | 28 | 28 |
| 10 | 5 | 3 |
| Total | 518 | 262 |

Column one of Table 7 shows the cluster number; column two, the number of genes; and column three, the number of genes correlated with steroid dose (CSD).

### Example 5

### RT-PCR Studies and Results

An exemplary RT-PCR study demonstrated the utility of steroid modulated nucleic acids and proteins in diagnosing and monitoring steroid responsiveness. Genes were chosen for the diagnostic set, and nucleic acid expression was reported as threshold cycle (CT) as measured using RT-PCR. The ratios of expression are calculated from the Ct values as 2^{(Ct(Control)-Ct(Rejection)}.

Gene expression was processed into a single score using voting, logistic regression or linear algorithms as detailed in Examples 1-3 of U.S. Ser. No. 11/433,191 and in Example 5 of U.S. Pat. No. 6,905,827, both incorporated by reference herein in their entirety. The diagnostic set of the 20 genes (11 formative, six normalization, three control) contained probes that were designed and tested as described in Examples 11 and 12, and RT-PCR, as described in Example 13, was conducted in triplicate RT-PCR reactions on samples from subjects on standard weaning protocols.

Of 104 index subjects, longitudinal gene expression profiles including post rejection and matched post non-rejection samples were available for 34 R subjects and 56 matched NR subjects at similar time points. The findings of the index study were extended to include samples and expression from an additional 192 consecutive subject encounters satisfying the inclusion criteria stated above. This set included samples from 118 new subjects and from 74 previous subjects and was used to estimate the prevalence of non-rejection in any 12 week period following sampling.

Longitudinal changes in expression from the index group were compared to corresponding scores for the larger group of 192 using repeated measure ANOVA. The probability that the transplant would not be rejected (negative predictive value) was calculated using EMB, rejection and non-rejection data. The Wald test was used with multivariate analysis to determine if, after controlling for clinical variables, the gene expression score remained a significant predictor of rejection.

Gene expression score, as calculated using a prediction algorithm, was found to be an independent predictor of future rejection at p=0.0266 when the clinical variables of recipient age, gender and race, panel reactive antibody, CMV serology status, and immunosuppression regimen (Wald test) were included. In fact, independent predictive value at p=0.015, was further enhanced in subjects≦180 days post-transplant.

Table 2 showed the p-value, as calculated using a t-test, for gene expression score and subject nucleic acid expression for 104 index samples, and for the subset of 74 samples <180 days post-transplant. Several of the individual genes shown in Table 2 showed differential expression associated with acute transplant rejection. Expression of IL1R2 decreased significantly (p=0.009, 1.6 fold) and PDCD1, increased significantly (p=0.032, 1.3 fold). In addition, IL1R2 (p<0.001) and FLT3 (p=0.024) demonstrated greater significance during the ≦180 day period and significant decreases in expression (2.3 and 1.6 fold, respectively) in subjects who progressed to rejection. During acute rejection, erythropoiesis genes, MIR and WDR40A, were up-regulated (both p=0.02), and FLT3 was down-regulated (p=0.03). The overall score was also significant using a Wilcoxon test for all subjects who progressed to rejection, p=0.011, and for those who did not progress, p<0.001. Those subjects who showed evidence of incipient rejection were placed immediately on anti-rejection therapy and subsequently showed a significant decrease in gene expression score (p<0.01).

The first RT-PCR study using a diagnostic set corresponding to the genes shown in Table 2 concluded: a) treatment of rejection with high dose steroids led to a statistically significant change in expression, b) low expression scores or a low value derived from evaluating expression scores with a prediction algorithm identified a group of subjects at very low risk for current and future rejection, and c) expression can be used to stratify subjects as to their risk of future rejection and lead to reduced number of cardiac biopsies.

The second RT-PCR study used PMBC samples from CARGO subjects and 33 nucleic acids/genes expressed in steroid modulated pathways. Analyses were based on all samples for which mRNA was available, 93 of 104 subjects in the all times post transplant group and 67 of 74 subjects in the ≦180 days post transplant group. Most of the nucleic acids came from the IL-1 and PDCD1 pathways and nucleic acids induced and expressed in T cells.

Table 3 shows the 33 genes grouped as to pathway, T cell associated, and other (TNF and NFKB1) and presented according to p-Value within the group. Differential expression of the genes is presented as fold change calculated as 2^{(mean controlCt-mean rejection Ct)}. Genes whose mRNA levels demonstrated a fold change >1 were up-regulated (increased) in subjects with rejection while those with a fold change <1 were down-regulated (decreased). P-value was based on t-test, and similar significance was obtained using the Mann-Whitney non-parametric test.

Using a p-value <0.05, five of the additional 33 genes tested supported the algorithm's steroid modulated constituents (IL1R2 and FLT3) while six, supported T-cell activation (PDCD1). Specifically, IL1R1, TSC22D3, FKBP5, THBS1 and CD163 showed significantly reduced expression; and ADA, GZMA, TRBC1, NFKB1, TNF and FLT3LG, significantly increased expression. Thus the methods of the invention and diagnostic sets of genes including but not limited to ADA, CD163, FKBP5, FLT3, FLT3LG, GZMA, IL1R1, IL1R2, ITGAM, NFKB1, PDCD1, THBS1, TNF, TRBC1 and TSC22D3 and selected from Tables 1-3 can be used for determining, diagnosing, evaluating, monitoring, or predicting disease activity, non-rejection, rejection, status of a transplant or of an immune disorder, steroid responsiveness, and treatment plan of a subject with a transplant or immune disorder.

Informative nucleic acids from the RT-PCR studies are listed in the table below as referenced to sequences in U.S. Pat. No. 6,905,827 or GenBank.

| GENE | SEQ ID NOs in USPN 6,905,827 |
|---|---|
| CD163 | 3857 |
| FKBP5 | 6299 |
| FLT3 | See GenBank sequence NM_004119 |
| IL1R2 | 4685 |
| ITGAM | 1981, 62 |
| THB1 | 4109, 264 |

### Example 6

### Prediction of Rejection or Non-Rejection

Quartile analysis was applied to the exemplary RT-PCR data for 74 subjects≦180 days post transplant. Subjects in the lowest quartile had expression scores≦20, and no subjects progressed to rejection in the subsequent 12 weeks (n=19). Subjects in the top quartile had expression scores≧30, and 58% of these subjects had rejection episodes (n=19) within 12 weeks of histological stability.

When this analysis was extended to the larger group of 192 representative consecutive samples, the incidence of subjects with expression scores≦20 were 33% of samples≦180 days post-transplant, and 98.9% of these remained rejection-free during the ensuing 12 weeks. Since the predictive value did not differ significantly by segmental time periods post transplant (30-60; 61-90; 90-180 days), a clinician can order 2-5 fewer EMBs for a subject with a low risk of rejection during the subsequent 12 weeks.

### Example 7

### Statistical Methods

The steroid modulated nucleic acids shown in the tables herein were identified in samples from subjects to whom steroids had been administered using at least one statistical method selected from various classification and prediction algorithms, software and programs. These methods include, but are not limited to, analysis of variance, classification and regression trees (Brieman et al. (1984) Classification and Regression Trees, Wadsworth, Belmont Calif.), cluster analysis including K-means clustering (MacQueen (1967) Proceedings of 5th Berkeley Symposium on Mathematical Statistics and Probability, University of California Press 1:281-297), Fisher Exact test, linear discriminatory analysis, logistic regression (Agresti (1996) An Introduction to Categorical Data Analysis. John Wiley and Sons Inc), multiple additive regression trees (Friedman (2002) Stanford University, Stanford Calif.), Mann-Whitney test, multivariate analysis, nearest shrunken centroids classifier (Tibshirani et al. (2002) PNAS 99:6567-6572), significance analysis of microarrays (Tusher et al. (2001) PNAS 98:5116-5121), one and two tailed T-tests, Wald test (Wald (1943) Trans Am Math Soc 54:426-482), Wilcoxon's signed ranks test, quartile analysis, and the like. Many of the above methods can be performed using SAS (SAS Institute, Cary N.C.) or Statistica (Statsoft, Tulsa Okla.). As noted in Example 1, the statistical methods applied to expression in order to chose a diagnostic set of nucleic acids or proteins are fully described in the Examples 1-3 of U.S. Ser. No. 11/433,191 and in Example 16 of U.S. Pat. No. 6,905,827, both incorporated by reference herein in their entirety.

### Example 8

### Preparation of Blood Samples, RNA Isolation from Lysate

Peripheral blood mononuclear cells (PBMC) were isolated from 8 mL venous blood using a VACUTAINER CPT tube (BD Biosciences (BD), San Jose Calif.) containing the anticoagulant sodium citrate, Ficoll Hypaque density fluid, and a thixotropic polyester gel. After the blood and tube components were mixed by inverting the tube 5-10 times, the tube was centrifuged, and mononuclear cells were collected from the fluid above the barrier layer. Approximately 2 mls of mononuclear cell suspension were transferred to a microfuge tube and centrifuged for 3 min at 16,000 rpm to pellet the cells. The pellet was resuspended and pipetted up and down in 1.8 ml of RLT lysis buffer (Qiagen, Chatsworth Calif.). Cell lysate was frozen and stored at −80 EC until total RNA was isolated.

After adding 5 ml of chloroform to the thawed lysate, the samples were vortexed and incubated at room temperature for 3 min. The aqueous layer was transferred to a new tube and purified using the RNeasy kit (Qiagen) according to the manufacturer's protocol. Isolated RNA was treated with DNAse on a QIASHREDDER column (Qiagen) and purified RNA was eluted in 50 μl of water. RNA purity was checked using the 2100 bioanalyzer and RNA 6000 microfluidics chips (Agilent Technologies, Palo Alto Calif.).

In the alternative, blood samples were collected in PAXgene Blood RNA tubes (Qiagen, Valencia Calif. and total RNA was purified using the PAXgene Blood RNA kit (Qiagen).

### Example 9

### cDNA Synthesis

cDNA was synthesized from purified RNA using reverse transcription with OLIGO-dT primers/random hexamers (Invitrogen, Carlsbad Calif.) at a final concentration of 0.5 ng/μl and 3 ng/μ, respectively. For the first strand reaction, 0.5 μg of mononuclear RNA and 1 μl of the OLIGO-dT/random hexamers (Invitrogen) were added to water in a reaction tube to a final volume of 11.5 μl. The tube was incubated at 70° C. for 10 min, chilled on ice, centrifuged, and 88.5 μl of first strand buffer mix (Invitrogen) was added to the tube.

The first strand buffer mix contained 1× first strand buffer, 10 mM DTT (Invitrogen), 0.5 mM dATP (New England Biolabs (NEB), Beverly Mass.), 0.5 mM dGTP (NEB), 0.5 mM dTTP (NEB), 0.5 mM dCTP (NEB), 200 U of SUPERSCRIPT RNAse H reverse transcriptase (Invitrogen), and 18 U of RNAGuard inhibitor (GE Healthcare (GEH), Piscataway N.J.). After the reaction was incubated at 42° C. for 90 min, the enzyme was heat-inactivated at 70° C. for 15 min. After adding 2 U of RNAse H (NEB) to the reaction tube, it was incubated at 37° C. for 20 min.

For second strand synthesis, 40 U of E. coli DNA polymerase (Invitrogen) and 2 U RNaseH (Invitrogen) were added to the previous reaction to bring the final volume to 150 μl. Salts and nucleotides were added to a final concentration of 20 mM Tris-HCl (pH 7.0; Fisher Scientific, Pittsburgh Pa.), 90 mM KCl (Teknova, Half Moon Bay Calif.), 4.6 mM MgCl2 (Teknova), 10 mM(NH₄)₂SO₄ (Fisher Scientific), 1× second strand buffer (Invitrogen), 0.266 mM dGTP, 0.266 mM dATP, 0.266 mM dTTP, and 0.266 mM dCTP.

After second strand synthesis for 150 min at 16° C., the cDNA was purified away from the enzymes, dNTPs, and buffers using phenol-chloroform extraction followed by ethanol precipitation in the presence of glycogen. Alternatively, the cDNA was purified on a QIAQUICK silica-gel column (Qiagen) followed by ethanol precipitation in the presence of glycogen. The cDNA was centrifuged at >10,000×g for 30 min. After the supernatant was aspirated, the pellet was washed with 150 μl of 70% ethanol and centrifuged. Following centrifugation, the supernatant was removed, and residual ethanol evaporated.

### Example 10

### Arrays

Arrays were used to identify steroid modulated genes in gene expression profiles from CARGO and LARGO subjects treated with steroids. In basic format, an array contains reagents specific for at least two nucleic acids or proteins, one that binds to a gene product of the invention and one that binds to a control gene product.

Nucleic Acid Arrays

Human Genome CGH 44A microarrays (Agilent Technologies) were used to determine differential expression. These Cy3/Cy5 chips contained 41,675 probes (60-mers) that represented most the genes found in REFSEQ database (NCBI); additional genes on the chip represented various controls. The chips were run as recommended by the manufacturer and scanned using an Agilent DNA microarray scanner. The data was extracted using Feature Extraction v 7.5 software (Agilent Technologies).

In the alternative, Affymetrix U133A Human GeneChips (Affymetrix, Santa Clara Calif.) with probe sets representing about 14,500 full length genes and 22,000 features were used according to the manuals and product inserts supplied by the manufacturer. Affymetrix Microarray Suite (MAS) v 5.0 software was used to generate expression values for each gene. To correct for slight differences in overall chip hybridization intensity and allow for comparison between samples, each chip was scaled to an overall intensity of 1500.

In another alternative, a low density array containing amplicons produced using probe sets for the nucleic acids selected from Tables 1-3 are harvested from PCR reactions, purified using Sephacryl-400 beads (GEH) and arrayed on a membrane. The membrane is UV irradiated, washed in 0.2% SDS at room temperature and rinsed three times in distilled water. Non-specific binding sites on the array are blocked by incubation in 0.2% casein in PBS for 30 min at 60° C., and the arrays are washed in 0.2% SDS and rinsed in distilled water prior to hybridization.

cDNAs are prepared from subject blood samples; diluted to a concentration of 40-50 ng in 45 μl TE buffer, denatured by heating to 100° C. for five min, and briefly centrifuged. The denatured cDNA is prepared using the Amersham CYSCRIBE first strand cDNA labeling kit (GEH) according to the manufacturer's instructions. The labeling reaction is stopped by adding 5 μl of 0.2M EDTA, and probe is purified from unincorporated nucleotides using a GFX Purification kit (GEH). The purified probe is heated to 100° C. for five min, cooled for two min on ice, and used in membrane-based hybridizations as described below.

Membranes are pre-hybridized in hybridization solution containing 1% Sarkosyl and 1× high phosphate buffer (0.5 M NaCl, 0.1 M Na₂HPO₄, 5 mM EDTA, pH 7) at 55° C. for two hr. The probe is diluted in 15 ml fresh hybridization solution and added to the membrane. The membrane is hybridized with the probe at 55° C. for 16 hr. Following hybridization, the membrane is washed once for 15 min at 25° C. in 1 mM Tris (pH 8.0) and 1% Sarkosyl and four times for 15 min each at 25° C. in 1 mM Tris (pH 8.0). To detect hybridization complexes, the membrane is exposed to x-ray film (Eastman Kodak) overnight at −70° C., developed, and examined visually or quantified using a scintillation counter (BeckmanCoulter, Fullerton Calif.).

Antibody arrays

Monoclonal antibodies are immobilized on a membrane, slide or dipstick or added to the wells of an ELISA plate using methods well known in the art. The array is incubated in the presence of serum or cell lysate until protein:antibody complexes are formed. The proteins encoded by genes or their splice variants are identified by the known position and labeling of the antibody that binds an epitope of that protein on the array. Quantification is normalized using the antibody:protein complex of various controls.

### Example 11

### Designing and Selecting Primers

Primers and probe sets were designed for the steroid modulated, normalization, and control genes using the PRIMER3 program (Whitehead Research Institute (WRI), Cambridge Mass.). Default values were used for all parameters but melting temperature (Tm). Tm was set between 71.7 and 73.7° C.; amplicon size, between 50 and 150 bases in length (optimum, about 100 bases); and primers or probes were allowed to be 36 nucleotides in length. Salt concentration, a critical parameter affecting the Tm of the probes and primers, was used at the default concentration, 50 mM.

The C source code for the PRIMER3 program was downloaded and compiled for use on machines running the Windows operating system (Microsoft, Redmond Wash.). To generate a number of potential primers, the program was run in batch mode from the command line using an input file that contained the sequences and the parameters for primer design. The first step was masked out repetitive sequences in the mRNA using the REPEATMASKER program (Institute for Systems Biology, University of Washington, Seattle Wash.). The second step masked out all known SNPs with allelic heterozygosity higher than 1% as annotated in the SNP database at NCBI (Bethesda Md.). The masked sequence was submitted to PRIMER3 using the parameters above, and the top pairs of primers were selected. Alternatively, the Primer3 program was used on the MIT website (Massachusetts Institute of Technology, Cambridge Mass.) to examine a specific region of the mRNA of a gene.

In the alternative, primer design software such as the web-based ProbeFinder software (Roche Diagnostics, Indianapolis Ind.), or provided by other suppliers of oligonucleotides, can be used to design primers and probes sets of the invention. The two step design process requires the name of the target organism and a sequence, gene name, or transcript ID number. The software will identify the Universal ProbeLibrary probes that will detect the nucleic acid. Primers were ordered from Roche Diagnostics, Integrated DNA Technologies (Coralville Iowa), or a similar commercial source.

### Example 12

### Testing of Primers and Probe Sets

Control genes: Experimental variation was monitored by adding one or more control genes to each array. β-actin, β-GUS, 18s ribosomal subunit, GAPDH, and β2-microglobulin were selected for low variability between samples and high expression across samples.

Primer Testing: Primers were tested at least once to see whether they produced an amplicon of the correct size and to determine their efficiency in a set of RT-PCR reactions using 5 serial dilutions of cDNA in water (1:10, 1:20, 1:40, 1:80, and 1:160). Each primer pair was tested on cDNA made from mononuclear cell RNA. The PCR reaction contained 1× RealTime-PCR buffer (Ambion, Austin Tex.), 2 mM MgCl2 (ABI), 0.2 mM dATP (NEB), 0.2 mM dTTP (NEB), 0.2 mM dCTP (NEB), 0.2 mM dGTP (NEB), 0.625 U AMPLITAQ Gold enzyme (ABI), 0.3 μM of each primer to be used (Sigma Genosys, The Woodlands Tex.), 5 μl of the reverse transcription reaction, and water added to a final volume of 19 μl.

Following 40 cycles of PCR, 10 μl of each PCR product was combined with Sybr Green dye at a final dilution of 1:72,000. Melt curves for each product were determined on a PRISM 7900HT Sequence detection system (ABI), and primer pairs yielding a product with one clean peak were chosen for further analysis. One μl of product from each probe set assay was examined by agarose gel electrophoresis or using a DNA 1000 chip kit and an Agilent 2100 bioanalyzer (Agilent Technologies). From primer design and the genomic sequence, the expected size of the amplicon was known. Only primer pairs showing amplification of the single desired product, and minimal amplification of contaminants, were used in assays.

### Example 13

### RT-PCR Assays and Analysis

CARGO: Ten μl RT-PCR reactions were performed to evaluate expression in the CARGO samples. TAQMAN Universal PCR Master mix (ABI) was aliquoted into light tight tubes, one for each gene. The primer pair for each gene was added to the tube of PCR master mix labeled for that gene. A FAM/TAMRA dual labeled TAQMAN probe (Biosearch Technologies, Novato Calif.) was added to each tube. Alternatively, different combinations of commercially available fluorescent reporter dyes and quenchers were used such that the absorption wavelength for the quencher matches the emission wavelength for the reporter. In the alternative, Universal ProbeLibrary probes (LNAs; Roche Diagnostics were substituted for TAQMAN probes.

Assays and Analysis: Each sample was dispensed into triplicate wells of a 384 well plate (ABI) for each primer pair. PCR reactions were run on the PRISM 7900HT Sequence Detection system (ABI) with the following conditions: 10 min at 95° C.; 40 cycles of 95° C. for 15 sec, 60° C. for 1 min. Sequence detection system v2.0 software (ABI) was used to analyze the fluorescent signal from each reaction. RT-PCR amplification product was measured as CT during the PCR reaction to observe amplification before any reagent became rate limiting. Threshold was set to a point where all of the reactions were in their linear phase of amplification. A lower CT indicated a higher amount of starting material (greater expression in the sample) since an earlier cycle number meant the threshold was crossed more quickly. A CT of less than 30 based on appropriate cDNA dilutions provided linear results for the blood samples from CARGO subjects. In the alternative, other technologies can be used to measure PCR product. Molecular beacons (Invitrogen) use FRET technology and disposable, microfluidic chip (Thermal Gradient, Pittsford N.Y.) employ silicon wafers to performed 30 cycle PCR in 4.4 min.

### Example 14

### Labeling Moieties

Labeling moieties can be used for detection of an antibody, nucleic acid or protein in any of the assays or diagnostic kits described herein. These labeling moieties include fluorescent, chemiluminescent, or chromogenic agents, cofactors, enzymes, inhibitors, magnetic particles, radionuclides, reporters/quenchers, substrates and the like that can be attached to or incorporated into the antibody, nucleic acid or protein. Visible labels and dyes include but are not limited to anthocyanins, avidin-biotin, β glucuronidase, biotin, BIODIPY, Coomassie blue, Cy3 and Cy5, 4,6-diamidino-2-phenylindole (DAPI), digoxigenin, ethidium bromide, FAM/TAMRA, FITC, fluorescein, gold, green fluorescent protein, horseradish peroxidase, lissamine, luciferase, phycoerythrin, reporter/quencher pairs (HEX/TAMRA, JOE/TAMRA, ROX/BHQ2, TAMRA/BHQ2, TET/BHQ1, VIC/BHQ1, and the like), rhodamine, spyro red, silver, streptavidin, and the like. Radioactive markers include radioactive forms of hydrogen, iodine, phosphorous, sulfur, and the like. They can be added to a primer or probe or to an antibody using standard protocols well know in the art and described in the specific nucleic acid and protein technologies described in Examples 9-14 and 16-17, respectively.

### Example 15

### Protein Expression

Adapter sequences for subcloning are added at either end of a coding region specific to a gene or a portion thereof and amplified using PCR. An epitope or affinity tag (6×his) or sequences for secretion from a cell can be added to the adapter sequence to facilitate purification and/or detection of the protein. The amplified cDNA is inserted into a shuttle or expression vector that can replicate in bacteria, insect, yeast, plant, or mammalian cells. Such vectors typically contain a promoter that operably links to the coding region, replication start sites, and antibiotic resistance or metabolite selection sequences.

The expression vector can be used in an in vitro translation system or to transfect cells. For example, Spodoptera frugiperda (Sf9) insect cells are infected with recombinant Autographica californica nuclear polyhedrosis virus (baculovirus). The polyhedrin gene is replaced with the cDNA by homologous recombination, and the polyhedrin promoter drives transcription. The protein is synthesized as a fusion protein with an affinity tag that enables purification.

Clones of transformed cells are analyzed to ensure that the inserted sequence is expressed. Once expression is verified, the cells are grown under selective conditions; and the protein is isolated from cells, or if secreted, from the growth media using chromatography, size exclusion chromatography, immunoaffinity chromatography, or other methods including cell fractionation, ion exchange, or selective precipitation.

The isolated and purified protein is then used as a reagent on an array or as an antigen to produce specific antibodies.

### Example 16

### Antibody Production and Testing

If antibodies are to be used as reagents, the sequence of the gene or splice variant is analyzed to determine regions of high immunogenicity (LASERGENE software; DNASTAR, Madison Wis.), and an appropriate oligopeptide is synthesized and conjugated to keyhole lympet hemocyanin (KLH; Sigma-Aldrich, St Louis Mo.).

Immunization

Rabbits are injected with the oligopeptide-KLH complexes in complete Freund=s adjuvant, and the resulting antisera is tested for specific recognition of the protein or fragments thereof. Antisera that react positively with the protein are affinity purified on a column containing beaded agarose resin to which the synthetic oligopeptide has been conjugated (SULFOLINK kit; Pierce Chemical, Rockford Ill.). The column is equilibrated using 12 ml IMMUNOPURE Gentle Binding buffer (Pierce Chemical). Three ml of rabbit antisera is combined with one ml of binding buffer and poured into the column. The column is capped (on the top and bottom), and antisera is allowed to bind with the oligopeptide by gentle shaking at room temperature for 30 min. The column is allowed to settle for 30 min, drained by gravity flow, and washed with 16 ml binding buffer (4×4 ml additions of buffer). The antibody is eluted in one ml fractions with IMMUNOPURE Gentle Elution buffer (Pierce Chemical), and absorbance at 280 nm is determined. Peak fractions are pooled and dialyzed against 50 mM Tris, pH 7.4, 100 mM NaCl, and 10% glycerol. After dialysis, the concentration of the purified antibody is determined using the BCA assay (Pierce Chemical), aliquoted, and frozen.

Electrophoresis and Blotting

Samples containing protein are mixed in 2× loading buffer, heated to 95° C. for 3-5 min and loaded on 4-12% NUPAGE Bis-Tris precast gel (Invitrogen). Unless indicated, equal amounts of total protein are loaded into each well. The gel is electrophoresed in 1× MES or MOPS running buffer (Invitrogen) at 200 V for approximately 45 min on an XCELL II apparatus (Invitrogen) until the RAINBOW marker (GEH) resolves and the dye front approaches the bottom of the gel. The gel is soaked in 1×transfer buffer (Invitrogen) with 10% methanol for a few minutes; and a PVDF membrane (Millipore, Billerica Mass.) is soaked in 100% methanol for a few seconds to activate it. The membrane, the gel, and supports are placed on the TRANSBLOT SD transfer apparatus (Biorad, Hercules Calif.) and a constant current of 350 mA is applied for 90 min.

Conjugation with Antibody and Visualization

After the proteins are transferred to the membrane, it is blocked in 5% (w/v) non-fat dry milk in 1× phosphate buffered saline (PBS) with 0.1% Tween 20 detergent (blocking buffer) on a rotary shaker for at least 1 hr at room temperature or at 4° C. overnight. After blocking, the buffer is removed, and 10 ml of primary antibody in blocking buffer is added and incubated on the rotary shaker for 1 hr at room temperature or overnight at 4° C. The membrane is washed 3 times for 10 min each with PBS-Tween (PBST), and secondary antibody, conjugated to horseradish peroxidase, is added at a 1:3000 dilution in 10 ml blocking buffer. The membrane and solution are shaken for 30 min at room temperature and washed three times for 10 min with PBST.

The wash solution is carefully removed, and the membrane is moistened with ECL+chemiluminescent detection system (GEH) and incubated for approximately 5 min. The membrane, protein side down, is placed on x-ray film (Eastman Kodak, Rochester N.Y.) and developed for approximately 30 seconds. Antibody:protein complexes are visualized and/or scanned and quantified.

What is claimed is:

1. A method of diagnosing or monitoring steroid responsiveness of a subject comprising:
a) detecting expression of a diagnostic set of at least two steroid modulated nucleic acids in a sample from the subject wherein the expression is correlated with steroid administration or dosage; and
b) applying at least one statistical method to the expression of the diagnostic set to diagnose or monitor steroid responsiveness of the subject.

2. The method of claim 1 wherein the diagnostic set further comprises at least one steroid modulated nucleic acid selected from each of at least two of the clusters of Table 1.

3. The method of claim 1 wherein the diagnostic set further comprises two or more steroid modulated nucleic acids selected from Tables 2 and Table 3.

4. The method of claim 1 wherein detecting the expression further comprises using hybridization or quantitative real-time polymerase chain reaction (RT-PCR).

5. The method of claim 1 wherein the sample further comprises a fluid obtained from the subject by any sampling means.

6. The method of claim 1 wherein the sample is blood containing peripheral blood mononuclear cells (PMBC).

7. The method of claim 1 wherein detecting expression of the diagnostic set of steroid modulated nucleic acids further comprises isolating RNA from the sample.

8. The method of claim 1 wherein the statistical method is K-means clustering or a prediction algorithm.

9. The method of claim 8 wherein K-means clustering produces clusters of genes that are correlated by p-value and their expression in a cell type or pathway.

10. The method of claim 8 wherein the prediction algorithm is selected from a linear algorithm, a logistic regression algorithm, and a voting algorithm and produces a single value or score.

11. The method of claim 1 wherein detecting expression of a diagnostic set further comprises selecting at least two oligonucleotides or a probe set to detect the expression of each nucleic acid of the diagnostic set.

12. A kit comprising the oligonucleotides or probe sets of claim 13.

13. The method of claim 1 wherein diagnosing or monitoring steroid responsiveness further comprises detecting the expression of nucleic acids encoding ADA, CD163, FKBP5, FLT3, FLT3LG, GZMA, IL1R1, IL1R2, ITGAM, NFKB1, PDCD1, THBS1, TNF, TRBC1 and TSC22D3.

14. A method for predicting rejection or non-rejection in a subject with a transplant comprising:
a) detecting expression of a diagnostic set of at least two steroid modulated nucleic acids in a sample from the subject wherein the expression of the steroid modulated nucleic acids correlates with transplant rejection or non-rejection; and
b) applying at least one statistical method to the expression of the diagnostic set of steroid modulated nucleic acids to predict rejection or non-rejection.

15. The method of claim 14 wherein the diagnostic set of steroid modulated nucleic acids further comprises two or more nucleic acids selected from Tables 1-3.

16. The method of claim 14 wherein the sample is PMBC.

17. The method of claim 14 wherein detecting expression of the diagnostic set of steroid modulated nucleic acids further comprises isolating RNA from the sample.

18. The method of claim 14 wherein detecting expression of the diagnostic set of steroid modulated nucleic acids further comprises using RT-PCR.

19. The method of claim 14 wherein the statistical method is a prediction algorithm that produces single value or score that correlates with rejection or non-rejection.

20. The method of claim 19 wherein the score that correlates with non-rejection is<20 and the score that correlates with rejection is>30.

21. The method of claim 14 wherein predicting rejection or non-rejection further comprises detecting the expression of a diagnostic set of steroid modulated nucleic acids encoding ADA, CD163, FKBP5, FLT3, FLT3LG, GZMA, IL1R1, IL1R2, ITGAM, NFKB1, PDCD1, THBS1, TNF, TRBC1 and TSC22D3.

22. A method of diagnosing or monitoring the status of a subject with a transplant comprising:
a) detecting expression of a diagnostic set of at least two steroid modulated nucleic acids in a sample from the subject wherein the expression is correlated with dysfunction or rejection of the transplant; and
b) applying at least one statistical method to the expression of the nucleic acids to monitor the status of the transplant.

23. The method of claim 22 wherein the diagnostic set further comprises two or more nucleic acids selected from Tables 1-3.

24. The method of claim 22 wherein the sample is PMBC.

25. The method of claim 22 wherein detecting expression further comprises isolating RNA from the sample.

26. The method of claim 22 wherein detecting expression further comprises using RT-PCR.

27. The method of claim 22 wherein the statistical method is a prediction algorithm that produces single value or score that correlates with the status of a subject with a transplant.

28. The method of claim 22 wherein diagnosing and monitoring the status of a subject with a transplant further comprises detecting the expression of a diagnostic set of steroid modulated nucleic acids encoding ADA, CD163, FKBP5, FLT3, FLT3LG, GZMA, IL1R1, IL1R2, ITGAM, NFKB1, PDCD1, THBS1, TNF, TRBC1 and TSC22D3.

29. A method for designing and monitoring a treatment plan for a subject with a transplant or an immune disorder comprising:
a) detecting expression of a diagnostic set of at least two steroid modulated nucleic acids in a sample from the subject wherein the expression correlates with the steroid responsiveness of the subject; and
b) using the expression of the diagnostic set of steroid modulated nucleic acids to design and monitor the treatment plan of the subject.

30. The method of claim 29 wherein the diagnostic set of steroid modulated nucleic acids further comprises two or more nucleic acids selected from Tables 1-3.

31. The method of claim 29 wherein the sample is PMBC.

32. The method of claim 29 wherein detecting expression further comprises isolating RNA from the sample.

33. The method of claim 29 wherein detecting expression further comprises using RT-PCR.

34. The method of claim 29 wherein the statistical method is a prediction algorithm.

35. The method of claim 29 wherein diagnosing and monitoring the treatment plan of a subject with a transplant or immune disorder further comprises detecting the expression of a diagnostic set of steroid modulated nucleic acids encoding ADA, CD163, FKBP5, FLT3, FLT3LG, GZMA, IL1R1, IL1R2, ITGAM, NFKB1, PDCD1, THBS1, TNF, TRBC1 and TSC22D3 whose expression correlates with steroid responsiveness of a subject.

36. The method of claim 29 wherein the transplant is selected from bone marrow, heart, kidney, liver, lung, pancreas, pancreatic islets, stem cells, xenotransplants, and artificial implants.

37. The method of claim 29 wherein the immune disorder is selected from cytomegalovirus infection, multiple sclerosis, and systemic lupus erythematosus.

38. A method for using primers and probe sets to detect steroid responsiveness of a subject with a transplant or an immune disorder comprising:
a) designing and generating primers or probe sets for nucleic acids whose expression is modulated by steroid administration or dosage; and
b) using RT-PCR and the primers or probe sets on a sample from the subject to detect steroid responsiveness.

39. The method of claim 38 wherein the nucleic acids whose expression is modulated by steroid administration or dosage are selected from Tables 1-3.
